SEQUENCE LISTING (3 sequences)

SEQ ID NO: 1 - DNA, 12 bp, synthetic construct
  source 1..12: mol_type = other DNA
gctagtggcgta

SEQ ID NO: 2 - DNA, 20 bp, synthetic construct
  source 1..20: mol_type = other DNA
tataaacttgtggtagttgg

SEQ ID NO: 3 - DNA, 20 bp, synthetic construct
  source 1..20: mol_type = other DNA
agctagtggcgtaggcaaga

(19) United States
(12) Patent Application Publication
(10) Pub. No.: US 2024/0263219 A1
(43) Pub. Date: Aug. 8, 2024

(54) METHODS AND COMPOSITIONS FOR IN SITU ANALYSIS OF VARIANT SEQUENCES

(71) Applicant: 10x Genomics, Inc., Pleasanton, CA (US)

(72) Inventor: Malte KÜHNEMUND, Stockholm (SE)

(21) Appl. No.: 18/405,884

(22) Filed: Jan. 5, 2024

Related U.S. Application Data
(60) Provisional application No. 63/437,600, filed on Jan. 6, 2023.

(51) Int. Cl. C12Q 1/6841; C12Q 1/682; C12Q 1/6827

(52) U.S. Cl. CPC C12Q 1/6841; C12Q 1/682; C12Q 1/6827

(57) ABSTRACT

The present disclosure relates in some aspects to methods for analyzing target nucleic acids in a biological sample. In some aspects, the presence, amount, and/or identity of a plurality of target nucleic acids comprising variant sequences are analyzed in situ in a sample. Also provided are oligonucleotides, sets of oligonucleotides, compositions, and kits for use in accordance with the methods.

### CROSS-REFERENCE TO RELATED APPLICATIONS

This application claims priority to U.S. Provisional Patent Application No. 63/437,600 filed on Jan. 6, 2023, entitled “METHODS AND COMPOSITIONS FOR IN SITU ANALYSIS OF VARIANT SEQUENCES,” which is herein incorporated by reference in its entirety for all purposes.

### REFERENCE TO AN ELECTRONIC SEQUENCE LISTING

The contents of the electronic sequence listing (202412017900SEQLIST.xml; Size: 3,701 bytes; and Date of Creation: Dec. 1, 2023) is herein incorporated by reference in its entirety.

### FIELD

The present disclosure relates in some aspects to methods and compositions for nucleic acid analysis in situ in biological samples, such as multiplex genotyping of single nucleotide differences in target nucleic acid molecules in situ in a cell or tissue sample.

### BACKGROUND

Methods are available for detecting nucleic acids present in a biological sample. For instance, advances in single molecule fluorescent in situ hybridization (smFISH) have enabled nanoscale-resolution imaging of RNA in cells and tissues. However, analysis of short sequences (e.g., single nucleotide differences such as single nucleotide polymorphisms (SNPs) or point mutations) on individual transcripts in situ has remained challenging. Improved methods for identifying these variant sequences and analyzing their spatial distribution in cell or tissue samples are needed. Provided herein are methods, compositions, and kits that address such and other needs.

### BRIEF SUMMARY

Certain methods for detecting variant sequences aim to discriminate a variant sequence (e.g., a particular SNP or point mutation) at the probe hybridization/ligation step. For instance, padlock probes with only one base difference in the arms (e.g., at the interrogatory nucleotide position of the probes) targeting a single nucleotide difference in target nucleic acids can compete with each other for a target sequence. The best matching probe can outcompete the other probes, become more stably hybridized to a target molecule, and can then be circularized using the target sequence as a ligation template. The ligated probe can be amplified in RCA and detected by various readout means. For instance, a barcode sequence in the padlock probe that corresponds to the matching variant sequence (e.g., a point mutation or a wildtype nucleotide) can be detected using hybridization based methods to detect the barcode sequence and identify whether an RCP is generated from a mutant or wildtype padlock probe.

Assays using probe hybridization/ligation to discriminate variant sequences can suffer from low specificity for multiple reasons, including properties of the ligase and/or the target nucleic acid. Low ligase fidelity can result in formation and detection of a ligation product (and subsequent amplicons of the ligation product), even when the sequence of interest does not match the interrogatory region of a probe, producing a high level of background or false positive results. For example, RNA templated ligases can tolerate some mismatches. In addition, ligases can have a strong base preference and probe end bias. Moreover, in the case of padlock probes targeting a single nucleotide difference, the single nucleotide is usually targeted by one arm, while the other arm covers a common region (e.g., a conserved region) among nucleic acid molecules containing different bases at the single nucleotide position. As a result, on the one hand, one arm of a first padlock probe could hybridize to the common region in a particular target nucleic acid molecule, but the other arm of the first padlock probe does not fully match the single nucleotide position in the particular target nucleic acid molecule. On the other hand, one arm of a second padlock probe could perfectly match the single nucleotide position in the target nucleic acid molecule, but the other arm of the second padlock probe cannot hybridize to the common region which is occupied by the first padlock probe or another probe. This is either ligated to a two-probe chimera that cannot be amplified by RCA, or probe hybridization can become unstable so that none of the padlock probes generates a ligation product and subsequently a detectable RCP, which can lead to a drop in detection efficiency. Improved methods for analyzing nucleic acids present in a biological sample, such as for in situ SNP genotyping, are needed.

In some embodiments, provided herein are methods comprising contacting target nucleic acids with a circularizable probe which, upon target hybridization, can be circularized by gap-filling and ligation, thereby incorporating target sequence information in the circularized probe. In some embodiments, before a splint oligonucleotide is ligated to fill the gap of a circularizable probe, an interrogatory region (e.g., an interrogatory nucleotide for an SNP or point mutation in the target nucleic acid) can be provided in the splint oligonucleotide, which is a separate molecule from the circularizable probe that is non-interrogatory (e.g., the circularizable probe can hybridize to conserved sequences outside the SNP or point mutation to be detected). For example, an interrogatory region of the splint oligonucleotide comprises a sequence that is complementary to the variant sequence (e.g., a SNP) in a target nucleic acid in the biological sample. Thus, splint oligonucleotides comprising different interrogatory regions can compete for hybridization to a target nucleic acid molecule, allowing the best matching interrogatory splint oligonucleotide to outcompete other splint oligonucleotides without interference from a non-interrogatory region that may decrease probe hybridization efficiency of the interrogatory region. Having an interrogatory region and a non-interrogatory region in physically separate molecules (e.g., one in splint oligonucleotide and the other in a gap-fill padlock probe) until they are ligated to each other may allow more efficient and effective competition among various interrogatory regions, increase hybridization specificity, and/or help reduce or avoid the probe competition problems associated with probes that are hybridized to target molecules as one piece.

In some embodiments, provided herein is a method for analyzing a biological sample, comprising: a) contacting the biological sample with a circularizable probe, wherein the circularizable probe comprises a first probe region and a second probe region that hybridize to a first target sequence and a second target sequence, respectively, in a target nucleic acid in the biological sample, wherein the first and second target sequences are separated by a gap sequence in the target nucleic acid, and wherein the gap sequence comprises a variant sequence among a plurality of different sequences; b) circularizing the circularizable probe to generate a circularized probe comprising a gap-filled region complementary to the gap sequence; c) generating a rolling circle amplification product (RCP) of the circularized probe in the biological sample, wherein the RCP comprises multiple copies of the gap sequence; d) contacting the biological sample with a plurality of nucleic acid probes, wherein each nucleic acid probe: i) comprises a hybridization region complementary to one of the plurality of different sequences; and ii) is detectably labeled or comprises a detectable region that directly or indirectly binds to a detection oligonucleotide comprising a detectable label, wherein a nucleic acid probe of the plurality of nucleic acid probes hybridizes to one of the multiple copies of the gap sequence in the RCP; and e) detecting a signal associated with the nucleic acid probe at a location in the biological sample, thereby identifying the variant sequence in the biological sample. In any of the embodiments herein, the plurality of different sequences can comprise a wildtype sequence and one or more variant sequences, for example, of a sequence polymorphism or mutations. In any of the embodiments herein, the variant sequence that is identified at the location in the biological sample can be a wildtype sequence, a mutation, or a variant sequence, for example, a variant of a sequence polymorphism (e.g., a major variant or a minor variant). In any of the embodiments herein, the variant sequence can be one or more nucleotides in length, and can be at a position within the gap sequence.

In any of the embodiments herein, the plurality of nucleic acid probes can be a plurality of intermediate probes, wherein each intermediate probe comprises: i) a hybridization region complementary to one of the plurality of different sequences; and ii) a detectable region that binds (e.g., hybridizes) to a detection oligonucleotide comprising a detectable label. In some embodiments, an intermediate probe of the plurality of intermediate probes hybridizes to one of the multiple copies of the gap sequence in the RCP.

In any of the embodiments herein, the plurality of nucleic acid probes can be a plurality of detectably labeled probes, wherein each detectably labeled probe comprises: i) a hybridization region complementary to one of the plurality of different sequences; and ii) a detectable label, such as a fluorescent label. In some embodiments, a detectably labeled probe of the plurality of detectably labeled probes hybridizes to one of the multiple copies of the gap sequence in the RCP.

In any of the embodiments herein, the plurality of nucleic acid probes can comprise a plurality of intermediate probes and a plurality of detectably labeled probes, wherein any one or more of the plurality of detectably labeled probes can directly or indirectly bind to one or more of the plurality of intermediate probes. In some embodiments, the plurality of nucleic acid probes form one or more branched structures, e.g., like branched hybridization complexes in a branched DNA (bDNA) assay. In some embodiments, in a branched complex, one or more of the plurality of detectably labeled probes hybridize to an intermediate probe of the plurality of intermediate probes. In some embodiments, in the branched complex, the intermediate probe hybridizes to another intermediate probe which in turn directly or indirectly bind to the RCP, e.g., at the gap sequence in the RCP.

In some embodiments, provided herein is a method for analyzing a biological sample, comprising contacting the biological sample with a circularizable probe comprising a first probe region and a second probe region that hybridize to a first target sequence and a second target sequence, respectively, in a target nucleic acid in the biological sample, where the first and second target sequences are separated by a gap sequence in the target nucleic acid, and where the gap sequence comprises a variant sequence among a plurality of different variant sequences. In some embodiments, the first target sequence and the second target sequence are common sequences shared by different molecules of the target nucleic acid, and the different molecules comprise gap sequences comprising different variant sequences flanked by the common first and second target sequences. In some embodiments, the method comprises circularizing the circularizable probe to generate a circularized probe comprising a gap-filled region complementary to the gap sequence. In some embodiments, a plurality of circularized probes are generated, each comprising a different gap-filled region comprising the complement of a different variant sequence. In some embodiments, the method comprises generating a rolling circle amplification product (RCP) of the circularized probe in the biological sample, where the RCP comprises multiple copies of the gap sequence. In some embodiments, a plurality of RCPs are generated, each comprising multiple copies of a different gap region comprising a different variant sequence.

In some embodiments, the method comprises contacting the biological sample with a plurality of intermediate probes, where each intermediate probe comprises: i) a hybridization region complementary to one of the plurality of different variant sequences; and ii) a detectable region that hybridizes to a detection oligonucleotide comprising a detectable label, where an intermediate probe of the plurality of intermediate probes hybridizes to one of the multiple copies of the gap sequence in the RCP. In some embodiments, the method comprises detecting signals associated with the detectable labels at locations in the biological sample, thereby detecting and/or identifying one or more of the plurality of different variant sequences in the biological sample.

In any of the embodiments herein, circularizing the circularizable probe can comprise contacting the biological sample with a library of splint oligonucleotides, where each splint oligonucleotide comprises: i) ligatable ends; and ii) a hybridization region complementary to one of the plurality of different variant sequences. In some embodiments, the method comprises ligating a splint oligonucleotide of the library of splint oligonucleotides that is complementary to the gap sequence to the circularizable probe, thereby circularizing the circularizable probe to generate the circularized probe. In some embodiments, the splint oligonucleotide comprises a 3′ hydroxyl group and a 5′ phosphate group. In some embodiments, each splint oligonucleotide in the library comprises a phosphate group at the 5′ end available for ligation. In some embodiments, the splint oligonucleotide comprises a 5′ flap and/or one or more RNA residues at and/or near its 3′ end.

In any of the embodiments herein, the splint oligonucleotide and/or the gap sequence can be between about 2 and about 40 nucleotides in length. In any of the embodiments herein, the splint oligonucleotide and/or the gap sequence can be between about 4 and about 24 nucleotides in length. In any of the embodiments herein, the splint oligonucleotide and/or the gap sequence can be between about 6 and about 18 nucleotides in length. In any of the embodiments herein, the variant sequence can be at the 3′ or 5′ end of the gap sequence. In any of the embodiments herein, the sequence complementary to the variant sequence can be at the 5′ or 3′ end of the splint oligonucleotide.

In any of the embodiments herein, the variant sequence can be at least 1, 2, 3, 4, 5, 6, 7, 8, 9, or more phosphodiester bonds from the 3′ or 5′ end of the gap sequence. In any of the embodiments herein, the sequence complementary to the variant sequence can be at least 1, 2, 3, 4, 5, 6, 7, 8, 9, or more phosphodiester bonds from the 5′ or 3′ end of the splint oligonucleotide. In any of the embodiments herein, the variant sequence can be at or near the central nucleotide(s) of the gap sequence. In any of the embodiments herein, the sequence complementary to the variant sequence can be at or near the central nucleotide(s) of the splint oligonucleotide.

In any of the embodiments herein, the variant sequence can comprise one or more nucleotides. In any of the embodiments herein, the variant sequence can be a nucleotide variation, a nucleotide polymorphism, a mutation, a substitution, an insertion, a deletion, a translocation, a duplication, an inversion, or a repetitive sequence. In some embodiments, the variant sequence is a single nucleotide variation.

In any of the embodiments herein, the variant sequence can be a single nucleotide. In any of the embodiments herein, the variant sequence can be a single nucleotide variation (SNV), single nucleotide polymorphism (SNP), a point mutation, a single nucleotide substitution, a single nucleotide insertion, or a single nucleotide deletion.

In any of the embodiments herein, the splint oligonucleotide can be ligated to the circularizable probe by a ligase using the target nucleic acid as a template. In any of the embodiments herein, the target nucleic acid can be an RNA or a DNA. In any of the embodiments herein, the ligase can have an RNA-templated ligase activity and/or a DNA-templated ligase activity.

In any of the embodiments herein, the library of splint oligonucleotides can comprise at least or about 2, at least or about 5, at least or about 10, at least or about 15, at least or about 20, at least or about 25, at least or about 30, at least or about 35, at least or about 40, at least or about 45, at least or about 50, or more splint oligonucleotides of different sequences.

In any of the embodiments herein, the molar concentration of the library of splint oligonucleotides is about equal to or about 2, about 4, about 8, about 10, or more times the molar concentration of the circularizable probe.

In any of the embodiments herein, the biological sample, the circularizable probe, and the library of splint oligonucleotides can be contacted with one another simultaneously or in any order. In any of the embodiments herein, the method can comprise washing the biological sample after contacting with the library of splint oligonucleotides. In any of the embodiments herein, the washing can be performed under less than stringent conditions.

In any of the embodiments herein, circularizing the circularizable probe can comprise: extending an end of the circularizable probe by a polymerase using the target nucleic acid as a template to generate an extended circularizable probe; and circularizing the extended circularizable probe to generate the circularized probe. In some embodiments, the target nucleic acid is an RNA. In some embodiments, the target nucleic acid is a DNA.

In any of the embodiments herein, the polymerase can have an RNA-templated polymerase activity and/or a DNA-templated polymerase activity. In any of the embodiments herein, the polymerase can be a reverse transcriptase. In any of the embodiments herein, the polymerase can incorporate one or more DNA residues and/or one or more RNA residues into the 3′ end of the circularizable probe to generate the extended circularizable probe. In any of the embodiments herein, the polymerase can have no or little strand displacement activity.

In any of the embodiments herein, the extended circularizable probe can be ligated by a ligase using the target nucleic acid as a template. In any of the embodiments herein, the target nucleic acid can be an RNA or a DNA. In any of the embodiments herein, the ligase can have an RNA-templated ligase activity and/or a DNA-templated ligase activity. In any of the embodiments herein, the extended circularizable probe can comprise one or more RNA residues at and/or near its 3′ end. The 3′ end (e.g., a 3′ ribonucleotide residue) can be ligated to the 5′ end of the extended circularizable probe to circularize the probe.

In any of the embodiments herein, the hybridization region in the intermediate probe can be between about 6 and about 18 nucleotides in length.

In any of the embodiments herein, the plurality of intermediate probes can comprise at least or about 2, at least or about 5, at least or about 10, at least or about 15, at least or about 20, at least or about 25, at least or about 30, at least or about 35, at least or about 40, at least or about 45, at least or about 50, or more intermediate probes comprising different hybridization regions.

In any of the embodiments herein, the detectable region can be in a 5′ overhang and/or a 3′ overhang of the intermediate probe. In any of the embodiments herein, the detectable label of the detection oligonucleotide can correspond to the hybridization region of the intermediate probe that hybridizes to the detection oligonucleotide. In some embodiments, the detectable label corresponds to hybridization regions of the same sequence. In some embodiments, the detectable label corresponds to hybridization regions of different sequences.

In any of the embodiments herein, one or more of the plurality of different variant sequences can be assigned a signal code sequence. In any of the embodiments herein, each different variant sequence can be assigned a unique signal code sequence, and identifying a variant sequence at a location in a sample can comprise detecting signals or absence thereof in sequential cycles to generate a detected signal code sequence, and comparing the detected signal code sequence with those in a codebook. By identifying a match (e.g., a best match among a few), the particular variant sequence that the corresponds to the detected signal code sequence can be identified at the location in the sample.

In any of the embodiments herein, detecting the one or more variant sequences can comprise: i) contacting the biological sample with a first intermediate probe and a first detection oligonucleotide to generate a first complex comprising the first intermediate probe hybridized to the gap sequence in the RCP and the first detection oligonucleotide hybridized to the first intermediate probe, where the first intermediate probe comprises: a first hybridization region complementary to the gap sequence, and a first detectable region, and where the first detection oligonucleotide comprises: a sequence complementary to the first detectable region, and a first detectable label; ii) imaging the biological sample to detect a first signal from the first detectable label, where the first signal corresponds to a first signal code in the signal code sequence. The contacting and imaging steps can be repeated one or more times using the same intermediate probe/detection oligonucleotide combination or a different intermediate probe/detection oligonucleotide combination. In any of the embodiments herein, detecting the one or more variant sequences can further comprise: iii) contacting the biological sample with a second intermediate probe and a second detection oligonucleotide to generate a second complex comprising the second intermediate probe hybridized to the gap sequence in the RCP and the second detection oligonucleotide hybridized to the second intermediate probe, where the second intermediate probe comprises: a second hybridization region complementary to the gap sequence, and a second detectable region, and where the second detection oligonucleotide comprises: a sequence complementary to the second detectable region, and a second detectable label; and iv) imaging the biological sample to detect a second signal from the second detectable label, where the second signal corresponds to a second signal code in the signal code sequence. The contacting and imaging steps can be repeated one or more times using the same intermediate probe/detection oligonucleotide combination (e.g., the first intermediate probe/first detection oligonucleotide, or the second intermediate probe/second detection oligonucleotide) or a different intermediate probe/detection oligonucleotide combination, to detect one or more subsequent signals each corresponding to a subsequent signal code in the signal code sequence. In some embodiments, the signal code sequence comprising at least the first signal code, the second signal code, and optionally a subsequent signal code is determined based on signals detected at a location in the biological sample, thereby detecting and/or identifying the one or more variant sequences of the target nucleic acid at the location in the biological sample. In some embodiments, the contacting in i) comprises contacting the biological sample with a first pool of intermediate probes and a universal pool of detection oligonucleotides, wherein the first pool of intermediate probes comprises the first intermediate probe and the universal pool of detection oligonucleotides comprises the first detection oligonucleotide and the second detection oligonucleotide, wherein each intermediate probe in the first pool of intermediate probes comprises (i) a hybridization region complementary to a gap sequence comprising one of the plurality of different sequences, and (ii) a detectable region complementary to a detection oligonucleotide of the universal pool of detection oligonucleotides, wherein the contacting in iii) comprises contacting the biological sample with a second pool of intermediate probes and the universal pool of detection oligonucleotides, wherein the second pool of intermediate probes comprises the second intermediate probe, and wherein each intermediate probe in the second pool of intermediate probes comprises (i) a hybridization region complementary to a gap sequence comprising one of the plurality of different sequences, and (ii) a detectable region complementary to a detection oligonucleotide of the universal pool of detection oligonucleotides.

In any of the embodiments herein, the biological sample can be contacted with a first pool of intermediate probes (e.g., the pool of intermediated probes for Cycle 1 in FIG. 2B) and a universal pool of detection oligonucleotides (e.g., DO1, DO2, and DO3 as shown in FIG. 2B), where the first pool of intermediate probes comprises the first intermediate probe and the universal pool of detection oligonucleotides comprises the first detection oligonucleotide and the second detection oligonucleotide. In some embodiments, each intermediate probe in the first pool of intermediate probes comprises (i) a hybridization region complementary to an identifier sequence (e.g., a nucleic acid analyte sequence such as a gap sequence comprising one of the plurality of different variant sequences, or a barcode sequence corresponding to the nucleic acid analyte sequence), and (ii) a detectable region complementary to a detection oligonucleotide of the universal pool of detection oligonucleotides. In any of the embodiments herein, the biological sample can be contacted with a second pool of intermediate probes (e.g., the pool of intermediated probes for Cycle 2 in FIG. 2B) and the universal pool of detection oligonucleotides (e.g., DO1, DO2, and DO3 as shown in FIG. 2B), where the second pool of intermediate probes comprises the second intermediate probe. In some embodiments, each intermediate probe in the second pool of intermediate probes comprises (i) a hybridization region complementary to an identifier sequence (e.g., a nucleic acid analyte sequence such as a gap sequence comprising one of the plurality of different variant sequences, or a barcode sequence corresponding to the nucleic acid analyte sequence), and (ii) a detectable region complementary to a detection oligonucleotide of the universal pool of detection oligonucleotides.

In any of the embodiments herein, the method can comprise identifying multiple different subsets of the plurality of different variant sequences of the target nucleic acid in the biological sample, where each subset is assigned a different signal code sequence. In some embodiments, each different subset independently comprises one or more different variant sequences. In some embodiments, a first subset comprises a wildtype sequence which is assigned a first signal code sequence, and a second subset comprises two, three, four, five, or more different mutant sequences which share the same assigned second signal code sequence. In some embodiments, for each different target nucleic acid, a first subset of the different variant sequences comprises a wildtype sequence which is assigned a signal code sequence which is different from that assigned to a second subset of the different variant sequences, where the second subset comprises two, three, four, five, or more different mutant sequences. For example, a first subset can be a KRAS wildtype sequence, and a second subset can include a plurality of KRAS mutant sequences in the 1-cycle detection exemplified in FIG. 2A, or in the multi-cycle detection exemplified in FIG. 2B. In some embodiments, the method comprises identifying multiple different variant sequences of the same target nucleic acid (e.g., KRAS) in the biological sample, where each subset (e.g., one or more different variant sequences) of the different variant sequences is assigned a different signal code sequence. For example, each subset can be a wildtype sequence or a different variant sequence (e.g., KRAS wildtype and KRAS mutants in the multi-cycle detection exemplified in FIG. 2A). In some embodiments, the method comprises identifying multiple different variant sequences of multiple different target nucleic acids (e.g., KRAS and TP53) in the biological sample. In some embodiments, for each different target nucleic acid a first subset of the different sequences comprises a wildtype sequence which is assigned a signal code sequence which is different from that assigned to a second subset of the different sequences. In some embodiments, each subset (e.g., one or more different variant sequences) of the different variant sequences of each different target nucleic acid is assigned a different signal code sequence as exemplified in FIG. 2B. In any of the embodiments herein, the number of different detection oligonucleotides in the universal pool can be two, three, four, five, six, or more. For example, the same pool of detection oligonucleotides DO1, DO2, DO3, and optionally DO4 can be contacted with the biological sample sequentially in Cycle 1, Cycle 2, . . . , and Cycle n.

In any of the embodiments herein, each different detection oligonucleotide can comprise a different detectable label corresponding to the sequence of the detection oligonucleotide. In any of the embodiments herein, each different detectable label corresponds to a different color in fluorescent microscopy. In some embodiments, detection oligonucleotide molecules of a first color hybridize to intermediate probes comprising hybridization regions of the same sequence. In some embodiments, the hybridization regions of the same sequence are complementary to a gap sequence comprising a wildtype sequence of the target nucleic acid. In some embodiments, detection oligonucleotide molecules of a second color hybridize to intermediate probes comprising hybridization regions of different sequences. In some embodiments, the hybridization regions of different sequences are complementary to gap sequences each comprising a mutant sequence of the target nucleic acid. For instance, in the 1-cycle detection shown in FIG. 2A, detection oligonucleotide molecules of a first color (“blue”) hybridize to intermediate probes comprising hybridization regions for the same wildtype KRAS sequence, whereas detection oligonucleotide molecules of a second color (“red”) hybridize to intermediate probes each comprising a hybridization region for a different mutant KRAS sequence.

In some embodiments, detection oligonucleotide molecules of the first color hybridize to intermediate probes comprising hybridization regions of the same sequence or different sequences. In some embodiments, the hybridization regions of the intermediate probes are complementary to gap sequences each comprising a wildtype or mutant sequence of the target nucleic acid or a wildtype or mutant sequence of another target nucleic acid. In some embodiments, detection oligonucleotide molecules of the second color hybridize to intermediate probes comprising hybridization regions of the same sequence or different sequences. In some embodiments, the hybridization regions of the intermediate probes are complementary to gap sequences each comprising a wildtype or mutant sequence of the target nucleic acid, a wildtype or mutant sequence of the another target nucleic acid, or a wildtype or mutant sequence of yet another target nucleic acid.

In any of the embodiments herein, the first pool of intermediate probes, the second pool of intermediate probes, and one or more subsequent pools of intermediate probes can be contacted with the biological sample in sequential cycles in a pre-determined sequence which corresponds to the signal code sequence assigned to the one or more variant sequences, where intermediate probes in the one or more subsequent pools each comprises (i) a hybridization region complementary to a gap sequence comprising one of the plurality of different variant sequences, and (ii) a detectable region complementary to a detection oligonucleotide of the universal pool of detection oligonucleotides. In any of the embodiments herein, the sequential cycles can comprise 2, 3, 4, 5, 6, 7, 8, 9, 10, or more cycles.

In some embodiments, provided herein is a method for analyzing a biological sample, comprising: a) contacting the biological sample with a circularizable probe, where the circularizable probe comprises a first probe region and a second probe region that hybridize to a first target sequence and a second target sequence, respectively, in a target nucleic acid in the biological sample, where the first and second target sequences are separated by a gap sequence in the target nucleic acid, and where the gap sequence comprises a variant sequence among a plurality of different variant sequences; b) contacting the biological sample with a library of splint oligonucleotides, where each splint oligonucleotide comprises: i) ligatable ends; and ii) a hybridization region complementary to one of the plurality of different variant sequences, where a splint oligonucleotide of the library of splint oligonucleotides that is complementary to the gap sequence is ligated to the circularizable probe, thereby circularizing the circularizable probe to generate a circularized probe comprising a gap-filled region complementary to the gap sequence; c) generating a rolling circle amplification product (RCP) of the circularized probe in the biological sample, where the RCP comprises multiple copies of the gap sequence; and d) detecting a sequence comprising the variant sequence in the gap sequence of the RCP at a location in the biological sample, thereby detecting the target nucleic acid comprising the variant sequence at the location in the biological sample. In some embodiments, the detecting of the sequence comprising the variant sequence in the gap sequence of the RCP is performed using a base-by-base sequencing method.

In some embodiments, provided herein is a method for analyzing a biological sample, comprising: a) contacting the biological sample with a circularizable probe, where the circularizable probe comprises a first probe region and a second probe region that hybridize to a first target sequence and a second target sequence, respectively, in a target nucleic acid in the biological sample, where the first and second target sequences are separated by a gap sequence in the target nucleic acid, and where the gap sequence comprises a variant sequence among a plurality of different variant sequences; b) contacting the biological sample with a library of splint oligonucleotides, where each splint oligonucleotide comprises: i) ligatable ends; and ii) a hybridization region complementary to one of the plurality of different variant sequences, where a splint oligonucleotide of the library of splint oligonucleotides that is complementary to the gap sequence is ligated to the circularizable probe, thereby circularizing the circularizable probe to generate a circularized probe comprising a gap-filled region complementary to the gap sequence; c) generating a rolling circle amplification product (RCP) of the circularized probe in the biological sample, where the RCP comprises multiple copies of the gap sequence; d) contacting the biological sample with a plurality of intermediate probes, where each intermediate probe comprises: i) a hybridization region complementary to one of the plurality of different variant sequences; and ii) a detectable region that hybridizes to a detection oligonucleotide comprising a detectable label, where an intermediate probe of the plurality of intermediate probes hybridizes to one of the multiple copies of the gap sequence in the RCP; and e) detecting signals associated with the detectable labels at locations in the biological sample, thereby identifying one or more of the plurality of different variant sequences in the biological sample. In some embodiments, the target nucleic acid is a target RNA.

In some embodiments, provided herein is a method for analyzing a biological sample, comprising: a) contacting the biological sample with a circularizable probe, where the circularizable probe comprises a first probe region and a second probe region that hybridize to a first target sequence and a second target sequence, respectively, in a target RNA in the biological sample, where the first and second target sequences are separated by a gap sequence in the target RNA, and where the gap sequence comprises a variant sequence among a plurality of different variant sequences; b) circularizing the circularizable probe to generate a circularized probe comprising a gap-filled region complementary to the gap sequence; c) generating a rolling circle amplification product (RCP) of the circularized probe in the biological sample, where the RCP comprises multiple copies of the gap sequence; and d) detecting a sequence comprising the variant sequence in the gap sequence of the RCP at a location in the biological sample, thereby detecting the target RNA comprising the variant sequence at the location in the biological sample.

In some embodiments, circularizing the circularizable probe comprises contacting the biological sample with a library of splint oligonucleotides, where each splint oligonucleotide comprises: i) ligatable ends; and ii) a hybridization region complementary to one of the plurality of different variant sequences, where a splint oligonucleotide of the library of splint oligonucleotides that is complementary to the gap sequence is ligated to the circularizable probe, thereby circularizing the circularizable probe to generate the circularized probe.

In some embodiments, circularizing the circularizable probe comprises extending an end of the circularizable probe by a polymerase using the target nucleic acid as a template to generate an extended circularizable probe; and circularizing the extended circularizable probe to generate the circularized probe.

In any of the embodiments herein, detecting the sequence comprising the variant sequence in the gap sequence of the RCP can comprise base-by-base sequencing of the sequence comprising the variant sequence.

In any of the embodiments herein, detecting the sequence comprising the variant sequence in the gap sequence of the RCP can comprise contacting the biological sample with a plurality of intermediate probes, where each intermediate probe comprises: i) a hybridization region complementary to one of the plurality of different variant sequences; and ii) a detectable region that hybridizes to a detection oligonucleotide comprising a detectable label, where an intermediate probe of the plurality of intermediate probes hybridizes to one of the multiple copies of the gap sequence in the RCP; and detecting signals associated with the detectable labels at locations in the biological sample.

In some embodiments, the circularized probe does not comprise a barcode sequence corresponding to the target nucleic acid or a sequence thereof. In some embodiments, the circularized probe comprises a barcode sequence corresponding to the target nucleic acid or a sequence thereof, where the barcode sequence is not complementary to the target nucleic acid or sequence thereof.

In any of the embodiments herein, the first and second probe regions can be common among a plurality of circularizable probes each targeting a molecule comprising a different variant sequence of the target nucleic acid. In any of the embodiments herein, the first probe region can be 3′ or 5′ to the second probe region in the circularizable probe. In any of the embodiments herein, the first probe region and the second probe region in the circularizable probe can be equal in length, or the first probe region can be shorter or longer than the second probe region. In any of the embodiments herein, the first probe region and/or the second probe region in the circularizable probe can be between about 5 and about 50 nucleotides in length. In any of the embodiments herein, the first probe region and/or the second probe region in the circularizable probe can be between about 15 and about 25 nucleotides in length. In any of the embodiments herein, the first probe region and/or the second probe region in the circularizable probe can comprise a 5′ flap and/or one or more RNA residues at and/or near its 3′ end.

In some embodiments, provided herein is a method for analyzing a biological sample, comprising: a) contacting the biological sample with a plurality of circularizable probes, wherein each circularizable probe comprises a first probe region and a second probe region that hybridize to a first target sequence and a second target sequence, respectively, in a target RNA in the biological sample, wherein the first and second target sequences are separated by a gap sequence in the target RNA, wherein the gap sequence in a wildtype molecule of the target RNA comprises a wildtype sequence, and wherein the gap sequence in a mutant molecule of the target RNA comprises a mutant sequence; b) contacting the biological sample with a library of splint oligonucleotides that compete for hybridization to the gap sequence, the library of splint oligonucleotides comprising: i) a wildtype splint oligonucleotide comprising ligatable ends and a hybridization region complementary to the gap sequence comprising the wildtype sequence of the target RNA, ii) a mutant splint oligonucleotide comprising ligatable ends and a hybridization region complementary to the gap sequence comprising the mutant sequence of the target RNA; c) ligating a splint oligonucleotide to a circularizable probe hybridized to a molecule of the target RNA, thereby circularizing the circularizable probe to generate a circularized probe comprising a gap-filled region complementary to the gap sequence in the target RNA; d) generating a rolling circle amplification product (RCP) of the circularized probe in the biological sample, wherein the RCP comprises multiple copies of the gap sequence; e) contacting the biological sample with a plurality of intermediate probes comprising: i) a wildtype intermediate probe comprising a wildtype hybridization region complementary to the gap sequence comprising the wildtype sequence of the target RNA, wherein the wildtype intermediate probe hybridizes to a detection oligonucleotide comprising a detectable label of a first color; and ii) a mutant intermediate probe comprising a mutant hybridization region complementary to the gap sequence comprising the mutant sequence of the target RNA, wherein the mutant intermediate probe hybridizes to a detection oligonucleotide comprising a detectable label of a second color, wherein the intermediate probe or the mutant probe hybridizes to the gap sequence in the RCP; and f) detecting a signal associated with the detectable label of the first color or the second color at a location in the biological sample, thereby identifying the wildtype sequence or the mutant sequence of the target RNA at the location in the biological sample.

In some embodiments, provided herein is a method for analyzing a biological sample, comprising: a) contacting the biological sample with a plurality of circularizable probes, where each circularizable probe comprises a first probe region and a second probe region that hybridize to a first target sequence and a second target sequence, respectively, in a target RNA in the biological sample, where the first and second target sequences are separated by a gap sequence in the target RNA, where the gap sequence in a wildtype molecule of the target RNA comprises a wildtype sequence, and where the gap sequence in a mutant molecule of the target RNA comprises a mutant sequence; b) contacting the biological sample with a library of splint oligonucleotides that compete for hybridization to the gap sequence, the library of splint oligonucleotides comprising: i) a wildtype splint oligonucleotide comprising ligatable ends and a hybridization region complementary to the gap sequence comprising the wildtype sequence of the target RNA, ii) a mutant splint oligonucleotide comprising ligatable ends and a hybridization region complementary to the gap sequence comprising the mutant sequence of the target RNA; c) ligating splint oligonucleotides to circularizable probes hybridized to molecules of the target RNA, thereby circularizing the circularizable probes to generate circularized probes each comprising a gap-filled region complementary to the gap sequence in the target RNA; d) generating rolling circle amplification products (RCPs) of the circularized probes in the biological sample, where each RCP comprises multiple copies of the gap sequence; e) contacting the biological sample with a plurality of intermediate probes comprising: i) a wildtype intermediate probe comprising a wildtype hybridization region complementary to the gap sequence comprising the wildtype sequence of the target RNA, where the wildtype intermediate probe hybridizes to a detection oligonucleotide comprising a detectable label of a first color; and ii) a mutant intermediate probe comprising a mutant hybridization region complementary to the gap sequence comprising the mutant sequence of the target RNA, where the mutant intermediate probe hybridizes to a detection oligonucleotide comprising a detectable label of a second color, where the intermediate probes hybridize to the gap sequences in the RCPs; and f) detecting signals associated with the detectable labels at locations in the biological sample, thereby identifying the wildtype sequence and/or the mutant sequence of the target RNA at the locations in the biological sample.

In any of the embodiments herein, the plurality of circularizable probes can comprise a common first target sequence and/or a common second target sequence. In any of the embodiments herein, the plurality of circularizable probes can hybridize to both the wildtype molecule and the mutant molecule of the target RNA. In any of the embodiments herein, the biological sample can comprise different molecules of the target RNA each comprising a mutant sequence of a plurality of different mutant sequences. In any of the embodiments herein, the library of splint oligonucleotides can comprise different mutant splint oligonucleotides each comprising a hybridization region complementary to a mutant sequence of a plurality of different mutant sequences. In any of the embodiments herein, the plurality of intermediate probes can comprise different mutant intermediate probes each comprising a mutant sequence of a plurality of different mutant sequences. In some embodiments, two or more of the different mutant intermediate probes hybridize to detection oligonucleotides comprising detectable labels of the same color. In some embodiments, two or more of the different mutant intermediate probes each hybridizes to a detection oligonucleotide comprising a detectable label of a different color. In some embodiments, the first color corresponds to the wildtype sequence and the second color corresponds to the mutant sequence, and the first and second colors are different. In some embodiments, the first color corresponds a signal code in a first signal code sequence corresponding to the wildtype sequence and the second color corresponds a signal code in a second signal code sequence corresponding to the mutant sequence, and the first and second colors are the same or different.

In any of the embodiments herein, the mutant sequence can be a single nucleotide. In any of the embodiments herein, the mutant sequence can be a single nucleotide variation (SNV), single nucleotide polymorphism (SNP), a point mutation, a single nucleotide substitution, a single nucleotide insertion, or a single nucleotide deletion.

In any of the embodiments herein, the biological sample can comprise target RNA molecules of different genes. In any of the embodiments herein, the library of splint oligonucleotides can comprise splint oligonucleotides comprising hybridization regions complementary to sequences in the target RNA molecules of the different genes. In any of the embodiments herein, the plurality of intermediate probes can comprise intermediate probes comprising the sequences in the target RNA molecules of the different genes.

In some embodiments, the biological sample is a cell or tissue sample. In any of the embodiments herein, the biological sample can be a cell or tissue sample comprising cells or cellular components. In any of the embodiments herein, the biological sample can be a tissue section. In any of the embodiments herein, the biological sample can be a formalin-fixed, paraffin-embedded (FFPE) sample, a frozen tissue sample, or a fresh tissue sample. In any of the embodiments herein, the biological sample can be fixed and/or permeabilized. In any of the embodiments herein, the biological sample can be crosslinked and/or embedded in a matrix. In any of the embodiments herein, the matrix can comprise a hydrogel. In any of the embodiments herein, the biological sample can be cleared.

In some embodiments, provided herein is a kit for analyzing a biological sample, comprising: a) a circularizable probe comprising a first probe region and a second probe region that hybridize to a first target sequence and a second target sequence, respectively, in a target nucleic acid in the biological sample, where the first and second target sequences are separated by a gap sequence in the target nucleic acid, and the gap sequence comprises a variant sequence among a plurality of different variant sequences; b) one or more reagents for circularizing the circularizable probe to generate a circularized probe comprising a gap-filled region complementary to the gap sequence; c) one or more reagents for generating a rolling circle amplification product (RCP) of the circularized probe, where the RCP comprises multiple copies of the gap sequence; d) a plurality of intermediate probes, where each intermediate probe comprises: i) a hybridization region complementary to one of the plurality of different variant sequences; and ii) a detectable region; and/or e) a plurality of detection oligonucleotides each comprising a detectable label and configured to hybridize the detectable region of an intermediate probe of the plurality of intermediate probes.

In some embodiments, provided herein is a kit for analyzing a biological sample, comprising: a) a circularizable probe comprising a first probe region and a second probe region that hybridize to a first target sequence and a second target sequence, respectively, in a target nucleic acid in the biological sample, where the first and second target sequences are separated by a gap sequence in the target nucleic acid, and the gap sequence comprises a variant sequence among a plurality of different variant sequences; b) a library of splint oligonucleotides, where each splint oligonucleotide comprises: i) ligatable ends; and ii) a hybridization region complementary to one of the plurality of different variant sequences, where a splint oligonucleotide of the library of splint oligonucleotides that is complementary to the gap sequence is ligated to the circularizable probe, thereby circularizing the circularizable probe to generate a circularized probe comprising a gap-filled region complementary to the gap sequence; and/or c) one or more reagents for generating a rolling circle amplification product (RCP) of the circularized probe, where the RCP comprises multiple copies of the gap sequence.

In some embodiments, provided herein is a kit for analyzing a biological sample, comprising: a) a circularizable probe comprising a first probe region and a second probe region that hybridize to a first target sequence and a second target sequence, respectively, in a target nucleic acid in the biological sample, where the first and second target sequences are separated by a gap sequence in the target nucleic acid, and the gap sequence comprises a variant sequence among a plurality of different variant sequences; b) a library of splint oligonucleotides, where each splint oligonucleotide comprises: i) ligatable ends; and ii) a hybridization region complementary to one of the plurality of different variant sequences, where a splint oligonucleotide of the library of splint oligonucleotides that is complementary to the gap sequence is ligated to the circularizable probe, thereby circularizing the circularizable probe to generate a circularized probe comprising a gap-filled region complementary to the gap sequence; c) one or more reagents for generating a rolling circle amplification product (RCP) of the circularized probe, where the RCP comprises multiple copies of the gap sequence; d) a plurality of intermediate probes, where each intermediate probe comprises: i) a hybridization region complementary to one of the plurality of different variant sequences; and ii) a detectable region; and/or e) a plurality of detection oligonucleotides each comprising a detectable label and configured to hybridize the detectable region of an intermediate probe of the plurality of intermediate probes.

In some embodiments, provided herein is a kit for analyzing a biological sample, comprising: a) a plurality of circularizable probes, where each circularizable probe comprises a first probe region and a second probe region that hybridize to a first target sequence and a second target sequence, respectively, in a target RNA in the biological sample, where the first and second target sequences are separated by a gap sequence in the target RNA, where the gap sequence in a wildtype molecule of the target RNA comprises a wildtype sequence, and where the gap sequence in a mutant molecule of the target RNA comprises a mutant sequence; b) a library of splint oligonucleotides comprising: i) a wildtype splint oligonucleotide comprising ligatable ends and a hybridization region complementary to the gap sequence comprising the wildtype sequence of the target RNA, ii) a mutant splint oligonucleotide comprising ligatable ends and a hybridization region complementary to the gap sequence comprising the mutant sequence of the target RNA; c) a plurality of intermediate probes comprising: i) a wildtype intermediate probe comprising a wildtype hybridization region complementary to the gap sequence comprising the wildtype sequence of the target RNA, and an overhang complementary to a detection oligonucleotide comprising a detectable label of a first color; and ii) a mutant intermediate probe comprising a mutant hybridization region complementary to the gap sequence comprising the mutant sequence of the target RNA, and an overhang complementary to a detection oligonucleotide comprising a detectable label of a second color; and/or d) a plurality of detection oligonucleotides comprising the detection oligonucleotide of the first color and the detection oligonucleotide of the second color.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The drawings illustrate certain embodiments of the features and advantages of this disclosure. These embodiments are not intended to limit the scope of the appended claims in any manner.

FIG. 1 shows an exemplary method of detecting variant sequences in a biological sample using a circularizable probe (e.g., a common circularizable probe that hybridizes to conserved regions of a target nucleic acid) and a library of splint oligonucleotides. The splint oligonucleotides compete with one another to hybridize to a region (e.g., a gap sequence) comprising a variant sequence (of one or more bases) in a target nucleic acid. The variant sequence can be wildtype or non-wildtype, or a major variant or minor variant, among a plurality of different possible sequences at the nucleotide position(s) in the region. The matching splint oligonucleotides are ligated to the common circularizable probe and the circularized probes are amplified via rolling circle amplification (RCA) and detected. For example, detection may be performed using detectably labeled probes that directly bind to a sequence of the RCA product or using a plurality of intermediate probes (e.g., that bind to a sequence of the RCA product) and detectably labeled probes (e.g., detection oligonucleotides, DOs) that directly or indirectly bind to the intermediate probes.

FIG. 2A shows exemplary methods where the detection using a plurality of intermediate probes and detectably labeled probes can be performed in one cycle or multiple cycles, using KRAS mutation detection as an example.

FIG. 2B shows an exemplary method of multiplex detection of multiple different genes X, Y, and Z, as well as multiple variants (e.g., wildtype and one or more mutants) of one or more genes.

FIG. 3A shows detection of a mutation of KRAS using circularizable probes and splint oligonucleotide ligation (Probe Set 2 and Probe Set 3) compared to a symmetrical circularizable probe (Probe Set 1, a 1-part symmetric circularizable probe with an interrogatory nucleotide on one arm; figure showing only the mutant probe) in two different cell lines, ME-180 (KRAS wildtype) and A549 (KRAS mutant).

FIG. 3B shows quantification of wildtype versus mutant (WT/MUT) KRAS RCP signals in ME-180 cells.

FIG. 3C shows quantification of mutant versus wildtype (MUT/WT) KRAS RCP signals in A549 cells.

FIG. 4A shows a schematic illustration of an example method of variant sequence detection in a “hot spot” region of KRAS using a common circularizable probe and a library of splint oligonucleotides, wherein each individual splint is complementary to wildtype KRAS (yellow), mutant KRAS (red), or other variant sequences of KRAS (orange).

FIG. 4B shows quantification of variant sequence detection in a “hot spot” region of KRAS using circularizable probes and splint oligonucleotide ligation from a library of splint oligonucleotides complementary to wildtype (G) KRAS (yellow), mutant (A) KRAS (red) and other variant sequences of KRAS (orange) within the “hot spot” region tested in two different cell lines, ME-180 (KRAS wildtype) and A549 (KRAS mutant).

### DETAILED DESCRIPTION

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

In some embodiments, provided herein are methods and compositions that reduce false positive signals (e.g., due to incorrect probe ligation) for the identification of variant sequences (e.g., SNPs) in situ in cell or tissue samples.

In some embodiments, provided herein is a method for in situ nucleic acid detection that may utilize but does not rely on discrimination of a variant sequence during target recognition (e.g., via probe hybridization to a nucleic acid analyte) and subsequent probe ligation (e.g., circularization of a circularizable probe using the nucleic acid analyte as template); instead, the method comprises variant sequence detection and identification (e.g., genotyping) in a readout stage, e.g., after probe hybridization, circularization, and generation of RCPs. In some embodiments, the readout stage comprises detecting a signal associated with the nucleic acid probe. In some embodiments, the readout can comprise identifying the sequence variations using sequencing-by-synthesis (SBS), sequencing-by-ligation (SBL), sequencing-by-binding (SBB) or avidity sequencing, or sequential hybridization of probes (e.g., intermediate probes and detectably labeled probes), or a combination thereof.

In some embodiments, a method disclosed herein comprises hybridizing a gap-fill circularizable probe or probe set (e.g., padlock probe) on conserved regions that flank a gap sequence in a nucleic acid target sequence. For instance, the conserved regions can be present in the majority or all RNA transcripts from the same gene (e.g., KRAS), and the gap sequences in the RNA transcripts may comprise one or more variant sequences (each of one or more bases), or regions to be interrogated (e.g., one or more SNPs) depending on the particular transcript. In some embodiments, the gap sequences comprise mutation hotspots. In some embodiments, a gap between the arms of a gap-fill circularizable probe or probe set (e.g., padlock probe) hybridized to conserved regions in the nucleic acid target can be filled using the gap sequence as a template, thereby incorporating sequence information regarding the variant sequence(s) from the nucleic acid target into the gap-filled circularizable probe or probe set (e.g., padlock probe). As used herein, a gap sequence can be an intervening sequence (of one or more bases) between a first target sequence and a second target sequence in a target nucleic acid, and the gap sequence can be linked to the first and second target sequences via one or more phosphodiester bonds. The first and second target sequences can be targets of a circularizable probe or probe set, such as arms of a gap-fill circularizable probe or probe set (e.g., padlock probe) that do not comprise a variant sequence or an interrogatory region. In some embodiments, the gap sequence comprises one or more variant sequences or regions to be interrogated, whereas the flanking first and second target sequences are constant or invariant among multiple target nucleic acid molecules.

In some embodiments, the splint oligonucleotides compete with one another for hybridization to a gap sequence that contains particular variant sequence(s). For instance, in the case of KRAS, mutations occur most frequently in 5 bases in codons 12 and 13. A library of splint oligonucleotides can be designed to cover all possible variants (or any subset thereof) in that region with a length of about 12 bases. In some embodiments, the splint oligonucleotides can be between about 6 and about 18 bases. In some embodiments, with short splint oligonucleotides, a single mismatch of one base, especially when the mismatch is in the middle of a short splint oligonucleotide, can decrease the stability of the splint oligonucleotide hybridization and the fully correct matching splint oligonucleotide is favored. Compared to a one-piece circularizable probe strategy where one arm of the probe matches a conserved region in the target sequence and a mismatch on the other arm (e.g., the arm containing an SNP-interrogatory nucleotide) does not affect the arm matching the conserved region, the splint oligonucleotide approach allows competition among splint oligonucleotides and dissociation of mismatched splint oligonucleotides prior to probe ligation.

In some embodiments, the ligation of the splint oligonucleotides onto the circularizable probes (e.g., gap-fill padlock probes) can be performed using RNA-templated ligation. In some embodiments, the ligation is performed after hybridization of the splint oligonucleotide library and removing splint oligonucleotides mismatched on target nucleic acids, and the method comprises ligating splint oligonucleotides matched with target nucleic acids to the circularizable probes. In some embodiments, the ligation is performed simultaneously with splint oligonucleotide hybridization, e.g., a ligase is present during splint oligonucleotide hybridization. In some embodiments, the splint oligonucleotide library and a plurality of circularizable probes are contacted with the sample simultaneously or in any order. In some embodiments, the sample is contacted with the splint oligonucleotide library and the plurality of circularizable probes at the same time, and the splint oligonucleotide library and the plurality of circularizable probes can be pre-mixed or not pre-mixed prior to contacting the sample. In some embodiments, the plurality of circularizable probes are hybridized to target nucleic acids in the sample before the splint oligonucleotide library is hybridized and ligated to the circularizable probes. In some embodiments, the splint oligonucleotide library is hybridized to target nucleic acids in the sample before the plurality of circularizable probes are hybridized and ligated to the splint oligonucleotides.

In some embodiments, one or more washes are performed between any of circularizable probe hybridization, splint oligonucleotide hybridization, and ligation. In some embodiments, any one or more of the washes can be stringent so that only completely complementary splint oligonucleotides remain bound to target nucleic acids after the wash(es). In some embodiments, any one or more of the washes can be performed under less than stringent conditions. In some embodiments, any one or more of the washes can be performed under extremely low stringency conditions, low stringency conditions, or medium stringency conditions.

For readout using base-by-base sequencing (e.g., SBS) or SBL, gene specific sequencing primers can be designed to target the RCPs in the conserved regions shortly before the mutation hotspot, such as a few bases (e.g., 3-5 bases or more) 5′ to the gap sequence in the RCPs, such that a conserved region and/or the adjacent gap sequence can be determined as identifier sequences to identify which gene the RCP corresponds to, and as the sequencing reaction continues and reads into the gap sequence (e.g., containing the hotspot), the variant sequence(s) can be readout base-by-base. Using the bases before the variant sequence(s) as identifier sequences, this approach can be multiplexed, e.g., multiple sequence variants (e.g., in mutation or SNP hotspots) of one or more genes or transcripts thereof can be identified at the same time. In some embodiments, between about 10 and about 20 bases in a conserved region and/or the adjacent gap sequence can be determined to cover most or all of the sequence variants in a hotspot and provide enough sequence information to identify the gene as well as the sequence variants therein.

In some embodiments, using sequential hybridization of probes (e.g., intermediate probes and detectably labeled probes that hybridize to the intermediate probes), a method disclosed herein can be used to distinguish one or more particular sequence variants from one or more other particular sequence variants, e.g., a major sequence variant (e.g., wildtype) versus multiple minor sequence variants (e.g., at least some mutants). In some embodiments, the readout can comprise a binary detection (e.g., “yes” or “no” for any one or more mutations). For example, one intermediate probe (e.g., an L-shaped probe) can be designed for the wildtype sequence (e.g., the intermediate probe can be detected using a blue fluorophore labeled probe, where a blue signal corresponds to the wildtype sequence) and a pool of intermediate probes (e.g., L-shaped probes) designed to cover some or all possible variant sequences. The pool of intermediate probes can be detected using the same detectably labeled probe, e.g., a red fluorophore labeled probe, that hybridizes to all of the intermediate probes in the pool, regardless of which sequence variant a particular intermediate probe is designed to match. In the particular example, the blue label corresponds to wildtype and the red label corresponds to “any variant” that is not wildtype. In some embodiments, the pool of intermediate probes for “any variant” plus the intermediate probe for wildtype can be identical as the splint oligonucleotide library, with the addition of an overhang to an splint oligonucleotide such that the overhang can hybridize to a detectably labeled probe. The intermediate probes can compete for the different copies of gap sequences in the RCPs, and at a particular RCP, generate a signal, either blue for wildtype or red for “any variant.” With this approach, one cycle is sufficient to determine whether the target nucleic acid molecule (e.g., a target RNA transcript) is wildtype or carries any mutation. This can be particularly useful in situations where it is not important to determine the exact variant (e.g., mutant) sequence, as often for applications such as cancer research or diagnostics, where the detection of the existence of a variant (e.g., any one or more of a few possible variants) is enough. One advantage of this approach is that the detection can be easily multiplexed to analyze a plurality of different target nucleic acids (e.g., transcripts from a panel of gene markers), for instance, for in situ genotyping.

In some embodiments, an intermediate probe comprises: i) a region comprising an at least partially degenerate sequence that hybridizes to an RCP comprising multiple copies of a gap sequence comprising a sequence variant, and ii) a region that hybridizes to a detectably labeled probe. The at least partially degenerate sequence can be degenerate except at at least the interrogatory position(s) complementary to the sequence variant. In some embodiments, the at least partially degenerate sequence is degenerate except at the interrogatory position complementary to a point mutation or SNP nucleotide in the gap sequence. A plurality of intermediate probes can be contacted with the sample comprising RCPs generated from gap-filled probes circularized from the circularizable probes, and the gap sequences comprising sequence variants in the RCPs can be identified by hybridization of detectably labeled probes to the intermediate probes and imaging the sample in sequential probe hybridization cycles. In some embodiments, the intermediate probes and detectably labeled probes in a cycle are dehybridized and removed (e.g., by washing the sample) after signal detection, and the intermediate probes and detectably labeled probes for the next cycle are applied to the sample to interrogate the next base position. For instance, for a hotspot having 5 nucleotides, 5 probe hybridization cycles can be performed to read all 5 bases. In some embodiments, the at least partially degenerate sequence in the intermediate probe can be fully degenerate.

In some embodiments, the method disclosed herein can be used for a gene with several loci where sequence variations and/or mutations occur with high frequency. For example, TP53 comprises 5 loci where mutations are highly frequent, and all 5 loci can be targeted with circularizable probes (in conjunction with gap-filling, e.g., by polymerization or by ligation of splint oligonucleotides). The readout can utilize a codebook comprising a code for TP53 wildtype and a cumulative code for detecting any one or more of the TP53 mutants, regardless of which one or more of the 5 loci contain the mutation(s) and which exact base is mutated at each mutation. If it is desirable to know which of the 5 loci is mutated, any one or more of the 5 loci and sequence variants therein can each have a corresponding code in the codebook, e.g., a code for wildtype TP53 locus 1, a code for “any variant” in TP53 locus 1, a code for wildtype TP53 locus 2, a code for variant 1 in TP53 locus 2, a code for variant 2 in TP53 locus 2, a code for variant 3 in TP53 locus 2, etc.

In some embodiments, one or more junctions for the gap-fill ligation is/are preferred ligation junction(s) for the ligase (e.g., a preferred ligation junction such as an AT, TA, GA, or AG ligation junctions for certain ligases). In some embodiments, the ligase has a neutral sequence preference for one or more junctions of the gap-fill ligation (e.g., TT, CT, CA, TC, AC, CC, TG, or AA ligation junctions for certain ligases). In some embodiments, the variant sequence of interest is not present at a sequence complementary to a preferred ligation junction for the ligase. In some embodiments, the variant sequence of interest is present at a sequence complementary to a non-preferred ligation junction (e.g., a CC, GT, GG, or GC junction is non-preferred for certain ligases). In some aspects, the use of gap-fill ligation facilitates improved detection of a variant sequence (e.g., a single nucleotide variation, single nucleotide polymorphism, a point mutation, a single nucleotide substitution, a single nucleotide insertion, or a single nucleotide deletion) that is not complementary to a preferred or neutral ligation junction in a probe. In some embodiments, the variant sequence of interest is not present at a sequence complementary to a preferred ligation junction for the ligase and one or more junctions for the gap-fill ligation is/are preferred ligation junction(s) for the ligase (e.g., a preferred ligation junction such as an AT, TA, GA, or AG ligation junctions for certain ligases). Without being bound by theory, gap-fill ligation facilitates use of ligation junctions at probe positions that are not complementary to the variant sequence itself, providing greater flexibility in probe design for efficient ligation.

### II. Nucleic Acid Analytes and Target Sequences

In some aspects, provided herein are methods and compositions for analysis of target nucleic acids. In some embodiments, the target nucleic acids comprise RNA. In some embodiments, the target nucleic acids comprise genomic DNA. In some embodiments, the target nucleic acids comprise cDNA. In some embodiments, one or more target nucleic acids each comprises a variant sequence of one or more nucleotides. In some embodiments, one or more target nucleic acids each comprises a single-nucleotide polymorphism (SNP). In some embodiments, one or more target nucleic acids each comprises is a single-nucleotide variant (SNV). In some embodiments, one or more target nucleic acids each comprises a single-nucleotide substitution. In some embodiments, one or more target nucleic acids each comprises a point mutation. In some embodiments, one or more target nucleic acids each comprises a single-nucleotide insertion. In some embodiments, one or more target nucleic acids each comprises a single-nucleotide deletion. In any of the embodiments herein, target genomic DNA, target RNA, and/or target cDNA comprising one or more sequence variants at one or more genomic loci can be analyzed as described herein. In some embodiments, target genomic DNA, target RNA, and/or target cDNA comprising one or more single-nucleotide differences (e.g., SNPs, SNVs, point mutations, etc.) at one or more genomic loci can be analyzed, and the identity of one or more single-nucleotide differences can be determined in situ in a sample.

In some embodiments, the target nucleic acid comprises a nucleotide variation, a nucleotide polymorphism, a mutation, a substitution, an insertion, a deletion, a translocation, a duplication, an inversion, and/or a repetitive sequence, in a variant sequence among a plurality of different sequences to be identified in situ in a biological sample. In some embodiments, the variant sequence is a single nucleotide, for instance, a single nucleotide variation (SNV), a single nucleotide polymorphism (SNP), a point mutation, a single nucleotide substitution, a single nucleotide insertion, or a single nucleotide deletion. In some embodiments, the variant sequence comprises multiple nucleotides, and each nucleotide can be independently at the position of an SNV, an SNP, a point mutation, a single nucleotide substitution, a single nucleotide insertion, or a single nucleotide deletion. In some embodiments, the target nucleic acid is an RNA, such as an miRNA or a transcript of an oncogene, a tumor suppressor gene, an immune gene, or an antigen receptor gene.

The methods, probes, and kits disclosed herein can be used to detect and analyze a wide variety of different analytes. Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to allow access of one or more reagents (e.g., probes for analyte detection) to the analytes in the cell or cell compartment or organelle.

Analytes of particular interest may include nucleic acid molecules (e.g., cellular nucleic acids), such as DNA (e.g. genomic DNA, cDNA, mitochondrial DNA, plastid DNA, viral DNA, etc.) and RNA (e.g. mRNA, microRNA, IRNA, snRNA, viral RNA, etc.), and synthetic and/or modified nucleic acid molecules (e.g. including nucleic acid domains comprising or consisting of synthetic or modified nucleotides such as LNA, PNA, morpholino, etc.).

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

Methods, probes, and kits disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte can comprise or be associated with a target sequence. In some embodiments, the target nucleic acid and the target sequence therein may be endogenous to the sample, generated in the sample, added to the sample, or associated with an analyte in the sample. In some embodiments, the target sequence is a single-stranded target sequence (e.g., a sequence in a rolling circle amplification product). In some embodiments, the target sequence is a single-stranded target sequence (e.g., in a probe bound directly or indirectly to the analyte). In some embodiments, the target sequence is a single-stranded target sequence in a primary probe that binds to an analyte of interest in the biological sample. In some embodiments, the target sequence is a single-stranded target sequence in an intermediate probe which directly or indirectly binds to a primary probe or product thereof, where the primary probe binds to an analyte of interest in the biological sample. In some embodiments, the target sequence is a single-stranded target sequence in a secondary probe that binds to the primary probe or product thereof. In some embodiments, the analytes comprise one or more single-stranded target sequences.

In some embodiments, provided herein are methods for in situ analysis of a target nucleic acid. In some embodiments, the in situ analysis comprises using different probes (e.g., splint oligonucleotides) to compete for hybridization to the target nucleic acid. In some embodiments, the splint oligonucleotides can be ligated to circularizable probes to circularize the probes. In some embodiments, the circularized probes can be amplified (e.g., using RCA) and the amplicons can be detected in situ.

In some embodiments, probes for in situ analysis can comprise circularizable probes that target common regions adjacent to hotspots for mutation. In some embodiments, the common regions flank a gap sequence in the target nucleic acid. In some embodiments, the gap sequence comprises one or more hotspots for mutation. In some embodiments, the gap sequence comprises a variant sequence among a plurality of different variant sequences. In some embodiments, gaps in the circularizable probes upon hybridization to their nucleic acid targets can be filled by polymerization. In some embodiments, the gaps can be filled by splint ligation, using a library of splint oligonucleotides that are diverse in sequences and comprise a plurality of possible variant sequences (e.g., possible mutations for the hotspots). In some embodiments, the library of splint oligonucleotides are incubated with the sample for hybridization to target nucleic acid molecules, allowing the best matching splint oligonucleotide to outcompete other splint oligonucleotides in the library. In some embodiments, after washing the sample, the best matching splint oligonucleotides can be ligated into the circularizable probes and the circularized probes can be amplified.

In some embodiments, the incorporation of variant sequence information into the probes can be hypothesis-free, requiring no prior knowledge of whether and which particular variant sequences are present in target nucleic acid molecules in a sample. In some embodiments, the gaps in circularizable probes hybridized to target nucleic acid molecules can be filled using polymerization (e.g., reverse transcription) templated on the gap sequences, followed by circularization of the extended circularizable probes (e.g., using a ligase having RNA-templated ligase activity).

In some embodiments, the incorporation of variant sequence information into the probes can take into consideration possible variant sequences covered by the gap sequences. In some embodiments, at least some of the gaps can be filled by ligating a library of splint oligonucleotides comprising complementary sequences to the possible variant sequences with the circularizable probes.

In some embodiments, amplicons (e.g., RCPs) comprising the gap sequences or complements thereof are detected in situ using sequencing-by-synthesis, sequencing-by-ligation, sequencing-by-binding, avidity sequencing, or sequential hybridization of probes. In some embodiments, the RCA products are sequentially contacted with pools of intermediate probes (e.g., nucleic acid probes), where each intermediate probe comprises (i) a hybridization sequence for recognition of a copy of a gap sequence or complement thereof in an RCP, which hybridization sequence can comprise the same sequence as a corresponding splint oligonucleotide in the library of splint oligonucleotides used to circularize the circularizable probes and (ii) a detectable region, such as an overhang sequence, for hybridization of a detectably labeled oligonucleotide.

### III. Circularizing the Circularizable Probes

In some embodiments, provided herein are molecules that can be circularized. In some embodiments, a circularizable probe or probe set is hybridized to a target nucleic acid molecule comprising a gap sequence which comprises one of multiple variant sequences, and the circularizable probe or probe set is circularized to generate a circularized probe comprising a gap-filled sequence complementary to the gap sequence in the target nucleic acid molecule. The circularized probe comprising at least portions of the complement of the gap sequence can be amplified (e.g., through RCA) and the amplification product can be detected in order to detect the variant sequence in the target nucleic acid molecule.

In some embodiments, provided herein is a method for analyzing a biological sample, the method comprising contacting the biological sample with a circularizable probe comprising a first probe region and a second probe region (e.g., 5′ and 3′ arms of a padlock probe) that hybridize to a first target sequence and a second target sequence, respectively, in a target nucleic acid (e.g., an RNA or cDNA) in the biological sample. In some embodiments, the first probe region and/or the second probe region in the circularizable probe comprises a 5′ flap and/or one or more RNA residues at and/or near its 3′ end. In some embodiments, the first and second probe regions are common among a plurality of circularizable probes that target a plurality of target nucleic acids that comprise different variant sequences. In some embodiments, each of the plurality of target nucleic acids comprises a common first target sequence (among the plurality of target nucleic acids) and a common second target sequence (among the plurality of target nucleic acids) that are complementary to the common first and second probe regions, respectively, among the plurality of circularizable probes. In some embodiments, the plurality of circularizable probes comprise molecules of the same nucleic acid sequence. In some embodiments, the plurality of circularizable probes comprise molecules of different nucleic acid sequences. In some embodiments, any two or more different nucleic acid sequences of the circularizable probes comprise i) the common first and second probe regions, and ii) different backbone sequences that are not complementary to target nucleic acid sequences. The different backbone sequences may each comprise a different identifier sequence. In some embodiments, the identifier sequence may identifier one or more circularizable probes from other circularizable probes. In some embodiments, the identifier sequence may comprise a barcode sequence. In some embodiments, the barcode sequence is associated with, corresponds to, and/or identifies a target nucleic acid or a sequence therein. In some embodiments, the barcode sequence is associated with, corresponds to, and/or identifies the plurality of target nucleic acids that comprise different variant sequences (e.g., a barcode sequence can correspond to target nucleic acids of a gene comprising various SNPs and/or point mutations in the gene).

In some embodiments, the first and second target sequences are separated by a gap sequence in the target nucleic acid. In some embodiments, the gap sequence can be about or at least 4, about or at least 6, about or at least 8, about or at least 10, about or at least 12, about or at least 14, about or at least 16, about or at least 18, about or at least 20, or more nucleotides in length. In some embodiments, the gap sequence comprises a variant sequence among a plurality of different variant sequences. In some embodiments, the plurality of circularizable probes that target a plurality of target nucleic acids that comprise different variant sequences do not hybridize to the gap sequences (which comprise the different variant sequences), and instead hybridize to common first and second target sequences that flank the gap sequences.

In some embodiments, upon hybridization to the target nucleic acid, the circularizable probe is circularized to generate a circularized probe comprising a gap-filled region complementary to the gap sequence. The gap-filled region can be generated using gap-filling by polymerization, or gap-fill splint ligation, or a combination thereof. In some embodiments, a rolling circle amplification product (RCP) of the circularized probe is generated in the biological sample, and the RCP comprises multiple copies of the gap sequence.

In some embodiments, the circularizable probe comprise a 5′ region and a 3′ region that hybridize to sequences adjacent to a hotspot for mutation in the target nucleic acid. In some embodiments, upon hybridization of a circularizable probe to the target nucleic acid molecule, the 3′ terminal nucleotide and the 5′ terminal nucleotide of the circularizable probe are not juxtaposed directly next to each other; as such, a ligase alone cannot catalyze the formation of a phosphodiester bond directly between the 5′ phosphate group of the 5′ terminal nucleotide and the 3′ hydroxyl group of the 3′ terminal nucleotide. In some embodiments, upon hybridization of a circularizable probe to the target nucleic acid molecule, the 3′ terminal nucleotide and the 5′ terminal nucleotide of the circularizable probe are separated from each other by a gap of between about 1 and about 40 nucleotides in length. In some embodiments, the gap is about 2, about 5, about 10, about 15, about 20, about 25, about 30, about 35, about 40, about 45, about 50, or of any integer (or range of integers) of nucleotides in between the indicated values. In some embodiments, the gap is no more than about 40 nucleotides in length. In some embodiments, the gap is no more than about 30 nucleotides in length. In some embodiments, the gap is about 6, about 8, about 10, about 12, about 14, about 16, about 18, about 20, about 22, about 24, or of any integer (or range of integers) of nucleotides in between the indicated values. In some embodiments, the gap is no more than about 10 nucleotides in length. In some embodiments, the gap is about 5 nucleotides in length.

In some embodiments, a circularizable probe or probe set disclosed herein does not comprise any nucleic acid barcode sequence. In some embodiments, circularizable probes or probe sets for hybridizing to multiple different target nucleic acids can comprise a common sequence that is not complementary to the target nucleic acids. For instance, the backbone sequences of a plurality of gap-fill padlock probes for detecting different variant sequences of a target nucleic acid can be a common backbone sequence. In other examples, the backbone sequences of a plurality of gap-fill padlock probes for detecting different target nucleic acids can be a common backbone sequence, and the arms of the gap-fill padlock probes can be different such that they can specifically hybridize to the target nucleic acids. In some embodiments, the backbone sequences of the plurality of gap-fill padlock probes do not contain any nucleic acid barcode sequence that uniquely corresponds to a particular target nucleic acid or a particular sequence variant thereof.

In some embodiments, a circularizable probe or probe set disclosed herein comprises one or more barcode sequences. In some embodiments, a circularizable probe or probe set comprises two or more barcode sequences. The barcode sequences, if present, may be of any length. If more than one barcode sequence is used, the barcode sequences may independently have the same or different lengths, such as at least 5, at least 10, at least 15, at least 20, at least 25, at least 30, at least 35, at least 40, at least 50 nucleotides in length. In some embodiments, the barcode sequence may be no more than 120, no more than 112, no more than 104, no more than 96, no more than 88, no more than 80, no more than 72, no more than 64, no more than 56, no more than 48, no more than 40, no more than 32, no more than 24, no more than 16, or no more than 8 nucleotides in length. Combinations of any of these are also possible, e.g., the barcode sequence may be between 5 and 10 nucleotides, between 8 and 15 nucleotides, etc.

The barcode sequence may be arbitrary or random. In certain cases, the barcode sequences are chosen so as to reduce or minimize homology with other components in a sample, e.g., such that the barcode sequences do not themselves bind to or hybridize with other nucleic acids suspected of being within the cell or other sample. In some embodiments, between a particular barcode sequence and another sequence (e.g., a cellular nucleic acid sequence in a sample or other barcode sequences in probes added to the sample), the homology may be less than 10%, less than 8%, less than 7%, less than 6%, less than 5%, less than 4%, less than 3%, less than 2%, or less than 1%. In some embodiments, the homology may be less than 20, 19, 18, 17, 16, 15, 14, 13, 12, 11, 10, 9, 8, 7, 6, 5, 4, 3, or 2 bases, and in some embodiments, the bases are consecutive bases.

In some embodiments, the number of distinct barcode sequences in a population of nucleic acid probes is less than the number of distinct targets of the nucleic acid probes, and yet the distinct targets may still be uniquely identified from one another, e.g., by encoding a probe with a different combination of barcode sequences. However, not all possible combinations of a given set of barcode sequences need be used. For instance, each probe may contain 1, 2, 3, 4, 5, 6, 7, 8, 9, 10, 11, 12, 13, 14, 15, etc. or more barcode sequences. In some embodiments, a population of nucleic acid probes may each contain the same number of barcode sequences, although in other cases, there may be different numbers of barcode sequences present on the various probes. In some embodiments, the barcode sequences or any subset thereof in the population of nucleic acid probes can be independently and/or combinatorially detected and/or decoded.

### A. Gap-Fill Polymerization

In some embodiments, a gap in a circularizable probe hybridized to the target nucleic acid molecule may be filled by extending a 3′ end of the circularizable probe or probe set. In some embodiments, a polymerase is used to extend the 3′ end using the target nucleic acid molecule as a template, thereby filling the gap using nucleotide sequence in the target nucleic acid molecule. In some embodiments, gap filling by the polymerase incorporates nucleotides residues into the circularizable probe or probe set, and the incorporated nucleotide sequence is complementary to the gap sequence or a portion thereof in the target nucleic acid molecule.

In some instances, the gap filling is performed using a polymerase (e.g., DNA polymerase) in the presence of appropriate dNTPs and other cofactors, under isothermal conditions or non-isothermal conditions. Exemplary DNA polymerases include but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

In some instances, the gap filling is performed using a DNA polymerase capable of incorporating at least about 25, at least about 50, at least about 100, at least about 125, at least about 150, at least about 175, at least about 200, at least about 225, at least about 250, at least about 300, at least about 400, at least about 500, at least about 600, or at least about 1,000 nucleotides in a single binding event before dissociating from the target nucleic acid molecule.

Incorporation of the correct nucleotides to a growing strand of DNA, as determined by the template, is known as sequence fidelity. In some embodiments, a high fidelity DNA polymerase is used for gap filling and examples include but are not limited to: Taq DNA polymerase, Phusion® High-Fidelity DNA Polymerase, KAPA Taq, KAPA Taq HotStart DNA Polymerase, KAPA HiFi, and/or Q5® High-Fidelity DNA Polymerase.

In some instances, the gap filling is performed using a polymerase having no or limited strand displacement activity, such that an extended 3′ region of the circularizable probe or probe set does not displace the 5′ region hybridized to the nucleic acid molecule. For example, T4 and T7 DNA Polymerases lack strand displacement activity and can be used for this purpose. In some embodiments, especially where the target nucleic acid is RNA, the polymerase can be a reverse transcriptase. Reverse transcriptases having reduced strand displacement activity can be used, see, e.g., Martin-Alonso et al., ACS Infect. Dis. 2020, 6, 5, 1140-1153, which is incorporated herein by reference in its entirety.

In some embodiments, the 3′ region of the circularizable probe extended by the polymerase can be juxtaposed to the 5′ region of the circularizable probe, forming a nick. In some embodiments, the ligation involves template dependent ligation, e.g., using the gap sequence in the target nucleic acid as template. In some embodiments, the ligation involves template independent ligation. The nick can be ligated using chemical ligation. In some embodiments, the chemical ligation involves click chemistry.

In some embodiments, the ligation involves enzymatic ligation. In some embodiments, the enzymatic ligation involves use of a ligase. In some aspects, the ligase used herein comprises an enzyme that is commonly used to join polynucleotides together or to join the ends of a single polynucleotide. In some aspects, the ligase used herein is a DNA ligase. In some aspects, the ligase used herein is an ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity. In some embodiments, the ligase is a ssDNA ligase. In some embodiments, the ssDNA ligase is a bacteriophage TS2126 RNA ligase or an archaebacterium RNA ligase or a variant or derivative thereof. In some embodiments, the ligase is Methanobacterium thermoautotrophicum RNA ligase 1, CircLigase™ I, CircLigase™ II, T4 RNA ligase 1, or T4 RNA ligase 2, or a variant or derivative thereof.

### B. Splint Oligonucleotide Ligation

In some embodiments, a gap in a circularizable probe hybridized to the target nucleic acid molecule may be filled by a splint oligonucleotide. In some embodiments, the splint oligonucleotide is ligated to a circularizable probe.

In some embodiments, the splint oligonucleotide can comprise a sequence complementary to a nucleotide variation, a nucleotide polymorphism, a mutation, a substitution, an insertion, a deletion, a translocation, a duplication, an inversion, and/or a repetitive sequence, for identifying a variant sequence among a plurality of different sequences in situ in a biological sample. In some embodiments, the splint oligonucleotide can comprise a sequence complementary to a single nucleotide, for instance, a single nucleotide variation (SNV), a single nucleotide polymorphism (SNP), a point mutation, a single nucleotide substitution, a single nucleotide insertion, or a single nucleotide deletion. In some embodiments, the splint oligonucleotide can comprise a sequence complementary to a sequence comprising multiple nucleotides, and each nucleotide can be independently at the position of an SNV, an SNP, a point mutation, a single nucleotide substitution, a single nucleotide insertion, or a single nucleotide deletion.

In some embodiments, provided herein is a library of splint oligonucleotides comprising i) a splint oligonucleotide comprising a sequence complementary to a nucleotide variation, a nucleotide polymorphism, a mutation, a substitution, an insertion, a deletion, a translocation, a duplication, an inversion, and/or a repetitive sequence, and ii) another splint oligonucleotide which does not comprise a sequence complementary to the nucleotide variation, nucleotide polymorphism, mutation, substitution, insertion, deletion, translocation, duplication, inversion, and/or repetitive sequence. In some embodiments, the library of splint oligonucleotides can comprise i) a splint oligonucleotide comprising a sequence complementary to a variant sequence or deletion or insertion, and ii) another splint oligonucleotide which does not comprise a sequence complementary to the variant sequence or deletion or insertion. For example, wildtype and variant splint oligonucleotides in the library, when contacted with the biological sample, can compete with one another for hybridization to a gap sequence comprising a variant sequence, and the complementary variant splint oligonucleotide can outcompete the wildtype splint oligonucleotide which is not complementary to the variant sequence (e.g., one or more nucleotides) in the gap sequence. The competition among splint oligonucleotides can allow the use of short (e.g., 2 nucleotides) splint oligonucleotides, while achieving specificity of splint oligonucleotide hybridization and/or ligation, for instance, when splint oligonucleotide hybridization and ligation are performed in the same reaction mix and/or the same reaction condition. In some embodiments, using a low hybridization temperature, less denaturation, and/or more co-factors such as Mg²⁺ or other factors that promote hybridization can enable the use of shorter splint oligonucleotides.

In some embodiments, upon hybridization to the target nucleic acid molecule, the 5′ terminal nucleotide of the splint oligonucleotide is adjacent to the 3′ terminal nucleotide of the circularizable probe, and the 3′ terminal nucleotide of the splint oligonucleotide is adjacent to the 5′ terminal nucleotide of the circularizable probe. In some embodiments, the 5′ terminal nucleotide of the splint oligonucleotide and the 3′ terminal nucleotide of the circularizable probe are separated by a nick or a gap of one or more nucleotides, e.g., 1, 2, 3, 4, 5, 6, 7, 8, 9, 10, or more nucleotides. In some embodiments, the 3′ terminal nucleotide of the splint oligonucleotide and the 5′ terminal nucleotide of the circularizable probe are separated by a nick or a gap of one or more nucleotides, e.g., 1, 2, 3, 4, 5, 6, 7, 8, 9, 10, or more nucleotides. The nick can be ligated using any suitable ligase disclosed herein, and the gap can be filled using any suitable ligase polymerase followed by ligation, for example, as described in Section III-A.

In some embodiments, upon hybridization to the target nucleic acid, the circularizable probe is circularized to generate a circularized probe comprising a gap-filled region complementary to the gap sequence.

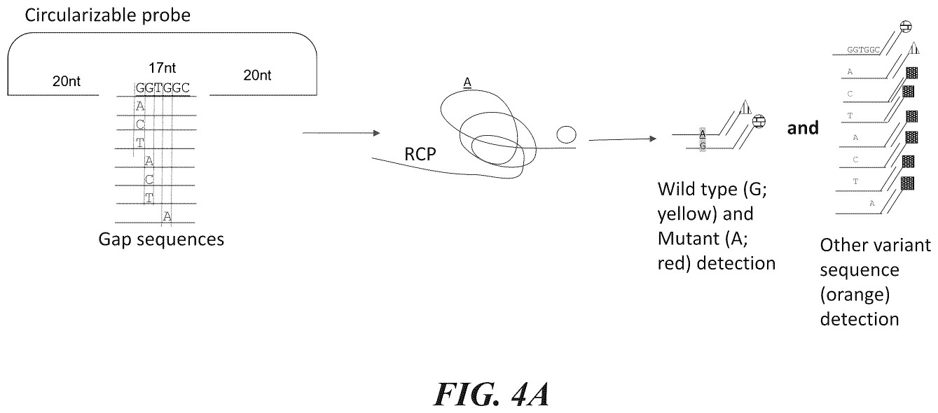

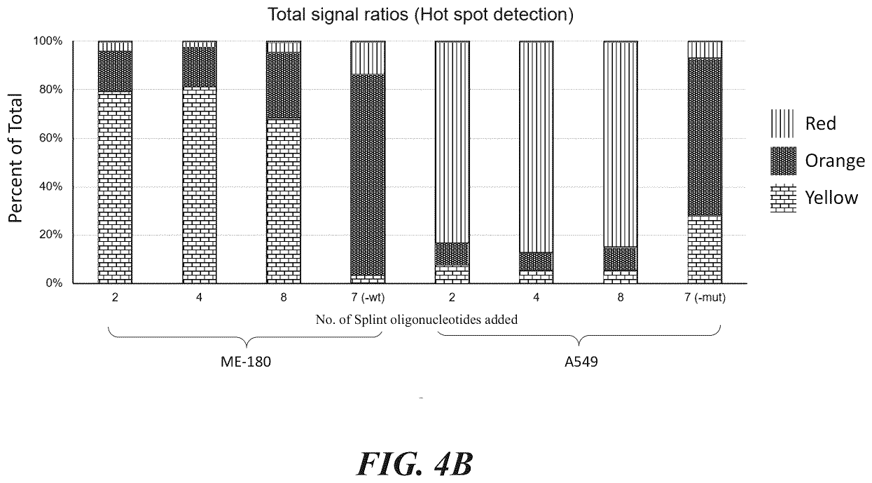

In some embodiments, the circularizable probe is hybridized to the target nucleic acid, followed by contacting the biological sample with a library of splint oligonucleotides that compete for hybridization to the target nucleic acid (e.g., hybridization to the gap sequence in the target nucleic acid). In some embodiments, the hybridization of a splint oligonucleotide to the target nucleic acid and the ligation of the splint oligonucleotide to the circularizable probe are performed sequentially, e.g., the splint oligonucleotide hybridization is performed in a reaction condition or reaction mix, and the splint oligonucleotide ligation is performed in a different reaction condition or different reaction mix. In some embodiments, the hybridization of a splint oligonucleotide to the target nucleic acid and the ligation of the splint oligonucleotide to the circularizable probe are performed in the same reaction condition or the same reaction mix. In some embodiments, any one or more of the splint oligonucleotides in the library can be 2 nucleotides or more in length. In some embodiments, the library of splint oligonucleotides is used to detect different variant sequences of a single target nucleic acid within the biological sample. In some embodiments, the different variant sequences of the single target nucleic acid are at different positions in the gap sequence. In some embodiments, a circularizable probe comprises a first probe region and a second probe region that hybridize to the common first target sequence and second target sequence shared by target nucleic acids comprising different variant sequences. For example, as shown in FIG. 4A, the library of splint oligonucleotides is used to detect wildtype KRAS, mutant KRAS, or other KRAS variant sequences within the same biological sample, while FIG. 4B shows example quantification of variant sequence detection within the biological sample using the library of splint oligonucleotides.

In some embodiments, the circularizable probe and the library of splint oligonucleotides can be contacted with the target nucleic acid at the same time, in the same reaction mix or separately. For example, the circularizable probe and the library of splint oligonucleotides can be premixed before contacting the biological sample with the mixture. In another example, two separate compositions comprising the circularizable probe and the library of splint oligonucleotides, respectively, can be contacted with the biological sample. In some embodiments, the hybridization of a splint oligonucleotide to the target nucleic acid and the ligation of the splint oligonucleotide to the circularizable probe are performed in the same reaction condition or the same reaction mix. In some embodiments, any one or more of the splint oligonucleotides in the library can be 2 nucleotides or more in length. In some embodiments, the library of splint oligonucleotides comprises at least or about 2, at least or about 5, at least or about 10, at least or about 15, at least or about 20, at least or about 25, at least or about 30, at least or about 35, at least or about 40, at least or about 45, at least or about 50, or more splint oligonucleotides of different sequences. In some embodiments, the molar concentration of the library of splint oligonucleotides is about equal to or about 2, about 4, about 8, about 10, or more times the molar concentration of the circularizable probe.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the splint oligonucleotide comprises a sequence complementary to the gap sequence in the target nucleic acid molecule. In some embodiments, the biological sample is contacted with a library of splint oligonucleotides. In some embodiments, the library comprises at least about 2, at least about 4, at least about 10, at least about 20, at least about 50, at least about 100, or more oligonucleotides of different sequences. In some embodiments, the sequence diversity of the splint oligonucleotides in the library is such that at least or about 80%, at least or about 85%, at least or about 90%, at least or about 95%, or about 100% of the possible variant sequences in the gap sequence of the target nucleic acid in a sample have corresponding splint oligonucleotides in the library, e.g., the splint oligonucleotides comprise sequences that are complementary to the variant sequences in the target nucleic acid.

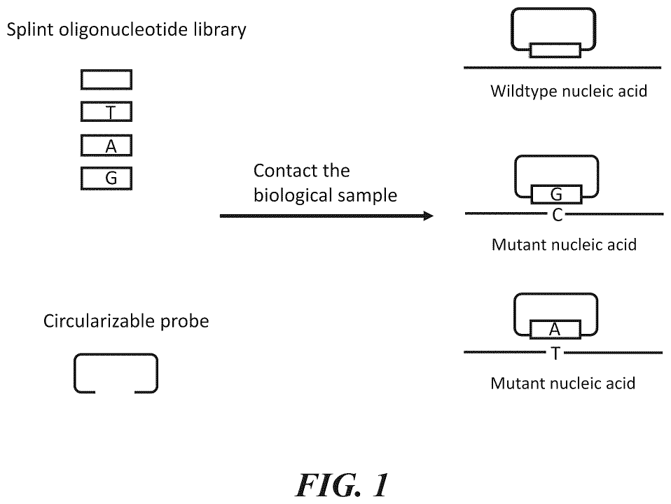

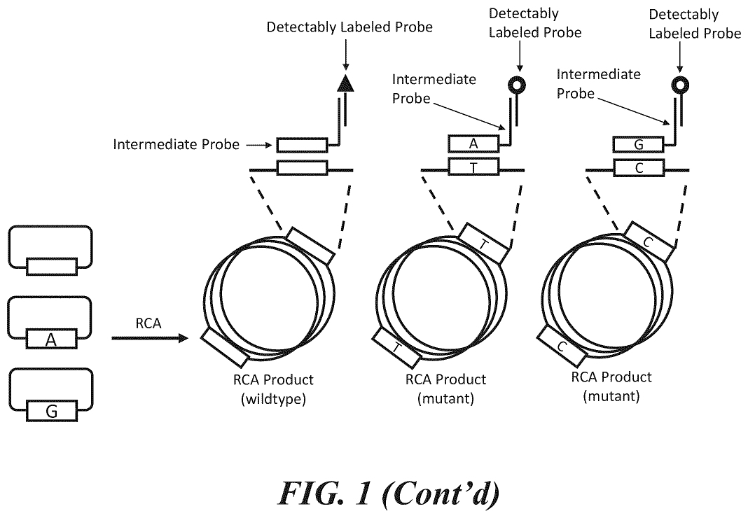

In some embodiments, the gap filling is performed under conditions permissive for specific hybridization of a splint oligonucleotide to its complementary sequence in the gap sequence in the target nucleic acid molecule, and/or specific hybridization of a circularizable probe or probe set to the target nucleic acid molecule. In some embodiments, the circularizable probe or probe set comprises hybridization regions that hybridize to the target nucleic acid molecule at sequences outside the gap sequence (e.g., at constant region sequences on the 5′ the 3′ of the hotspot for mutation), whereas the variant sequences in the gap sequence are complementary to the splint oligonucleotides (e.g., wildtype or mutant) in the library, as shown in FIG. 1. In some embodiments, the circularized probe is amplified by RCA (e.g., as described in Section IV), and the RCA product comprises multiple copies of the gap sequence in the target nucleic acid, as shown in FIG. 1. In some embodiments, a sequence in the gap sequence in the RCA product is determined in situ, e.g., by sequencing the gap sequence using SBS, SBB, SBH, SBL, or sequential hybridization of probes, etc., as described in Section V.

In some embodiments, the splint oligonucleotides can be between about 6 and about 24 nucleotides in length. In some embodiments, any one or more of the splint oligonucleotides in the library can be about 6, about 8, about 10, about 12, about 14, about 16, about 18, about 20, about 22, or about 24 nucleotides in length. Any two or more of the splint oligonucleotides in the library can have the same length or different lengths. In some embodiments, the splint oligonucleotides in the library can be of the same length.

In some embodiments, the variant sequence can be at least 1, 2, 3, 4, 5, 6, 7, 8, 9, or more phosphodiester bonds from the 3′ or 5′ end of the gap sequence. In some embodiments, the sequence complementary to the variant sequence can be at least 1, 2, 3, 4, 5, 6, 7, 8, 9, or more phosphodiester bonds from the 5′ or 3′ end of the splint oligonucleotide. In some embodiments, the variant sequence can be at or near the central nucleotide(s) of the gap sequence. In some embodiments, the sequence complementary to the variant sequence can be at or near the central nucleotide(s) of the splint oligonucleotide. In some embodiments, the sequence complementary to the variant sequence is at or near the central 1, 2, 3, 4, or 5 nucleotide(s) of the splint oligonucleotide. In some embodiments, the sequence complementary to the variant sequence can be no more than 1, no more than 2, no more than 3, no more than 4, no more than 5, or no more than 6 nucleotides from the central nucleotide(s) of the splint oligonucleotide.

### IV. Rolling Circle Amplification (RCA)

Following formation of the circularized probe, in some instances, a primer oligonucleotide is added for amplification. In some instances, the primer oligonucleotide is added with the circularizable probe or probe set. In some instances, the primer oligonucleotide is added before or after the circularizable probe or probe set is contacted with the sample. In some instances, the primer oligonucleotide for amplification of the circularized probe may comprise a sequence complementary to a target nucleic acid, as well as a sequence complementary to the circularizable probe that hybridizes to the target nucleic acid. In some embodiments, amplification of the circularized probe or probe set is primed by the target nucleic acid. In some embodiments, a washing step is performed to remove any unbound probes, primers, etc. In some embodiments, the wash is a stringency wash. Washing steps can be performed at any point during the process to remove non-specifically bound probes, probes that have ligated, etc.

A primer oligonucleotide for amplification of the circularized probe can comprise a single-stranded nucleic acid sequence having a 3′ end that can be used as a substrate for a nucleic acid polymerase in a nucleic acid extension reaction. The primer oligonucleotide can comprise both RNA nucleotides and DNA nucleotides (e.g., in a random or designed pattern). The primer oligonucleotide can also comprise other natural or synthetic nucleotides described herein that can have additional functionality. The primer oligonucleotide can be about 6 bases to about 100 bases, such as about 25 bases.

In some embodiments, amplification of the circularized probe or probe set is primed by the target nucleic acid (e.g., target RNA). The target nucleic acid can optionally be immobilized in the biological sample. In some embodiments, the target RNA is cleaved by an enzyme (e.g., RNase H). In some embodiments, the target nucleic acid is cleaved at a position downstream of the first target sequence and a second target sequences bound to the circularized probe or probe set. In some aspects, the methods disclosed herein allow targeting of RNase H activity to a particular region in a target RNA that is adjacent to or overlapping with a target sequence for a probe or probe set. For example, a nucleic acid oligonucleotide is designed to hybridize to a complementary oligonucleotide hybridization region in the target RNA. In some embodiments, a nucleic acid oligonucleotide is used to provide a DNA-RNA duplex for RNase H cleavage of the target RNA in the DNA-RNA duplex. In some embodiments, the oligonucleotide binds to the target RNA at a position that overlaps with the target sequence of the probe or probe set by about 1 to about 20 nucleotides or by about 8 to about 10 nucleotides. The cleaved target RNA itself can then be used to prime RCA of the circular probe generated from a circularized probe or probe set (e.g., target-primed RCA). In some cases, a plurality of nucleic acid oligonucleotides can be used to perform target-primed RCA for a plurality of different target nucleic acids.

In any of the embodiments herein, the biological sample is contacted with the RNase H (and optionally with the nucleic acid oligonucleotide) before or during formation of the circularized gap-filled probe or probe set (e.g., as described in Section III). In some embodiments, the biological sample is contacted with the oligonucleotide and with the RNase H simultaneously or sequentially (in either order) before contacting the sample with the probe or probe set. In any of the embodiments herein, the biological sample can be contacted with the RNase H (and optionally with the nucleic acid oligonucleotide) after formation of the circularized probe or probe set. In any of the embodiments herein, the RNase H comprises an RNase H1 and/or an RNAse H2. In some embodiments, RNase inactivating agents or inhibitors are added to the sample after cleaving the target RNA.

In some instances, upon addition of a DNA polymerase in the presence of appropriate dNTP precursors and other cofactors, the amplification primer is elongated by replication of multiple copies of the template. The amplification step can utilize isothermal amplification or non-isothermal amplification. In some embodiments, after the formation of the hybridization complex and any subsequent circularization (such as ligation of, e.g., a circularizable probe), the circularized probe is rolling-circle amplified to generate a RCA product (e.g., amplicon) containing multiple copies of the sequence of the circularized probe.

In some embodiments, RCPs are generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof. In some embodiments, the polymerase is Phi29 DNA polymerase.

In some embodiments, the polymerase comprises a modified recombinant Phi29-type polymerase. In some embodiments, the polymerase comprises a modified recombinant Phi29, B103, GA-1, PZA, Phi15, BS32, M2Y, Nf, G1, Cp-1, PRD1, PZE, SF5, Cp-5, Cp-7, PR4, PR5, PR722, or L17 polymerase. In some embodiments, the polymerase comprises a modified recombinant DNA polymerase having at least one amino acid substitution or combination of substitutions as compared to a wildtype Phi29 polymerase. Exemplary polymerases are described in U.S. Pat. Nos. 8,257,954; 8,133,672; 8,343,746; 8,658,365; 8,921,086; and 9,279,155, all of which are herein incorporated by reference. In some embodiments, the polymerase is not directly or indirectly immobilized to a substrate, such as a bead or planar substrate (e.g., glass slide), prior to contacting a sample, although the sample may be immobilized on a substrate.

In some embodiments, the amplification is performed at a temperature between or between about 20° C. and about 60° C. In some embodiments, the amplification is performed at a temperature between or between about 30° C. and about 40° ° C. In some aspects, the amplification step, such as the rolling circle amplification (RCA) is performed at a temperature between at or about 25° C. and at or about 50° C., such as at or about 25° C., 27° ° C., 29° C., 31° C., 33° C., 35° C., 37° ° C., 39° C., 41° C., 43° ° C., 45° C., 47° C., or 49° ° C.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide reacts with an acrylic acid N-hydroxysuccinimide moiety. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification. In some embodiments, the modified nucleotides comprises base modifications, such as azide and/or alkyne base modifications, dibenzylcyclooctyl (DBCO) modifications, vinyl modifications, trans-Cyclooctene (TCO), and so on.

In some embodiments, the primer extension reaction mixture can comprise a deoxynucleoside triphosphate (dNTP) or derivative, variant, or analogue thereof. In some embodiments, the primer extension reaction mixture can comprise a catalytic cofactor of the polymerase. In any of the preceding embodiments, the primer extension reaction mixture can comprise a catalytic di-cation, such as Mg²⁺ and/or Mn²⁺.

In some aspects, the amplification product (e.g., RCA product) can be anchored to a polymer matrix. The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products (e.g., RCA products) are copolymerized and/or covalently attached to the surrounding matrix thereby preserving their spatial relationship and any information inherent thereto. In some embodiments, the RCA products are generated from DNA or RNA within a cell embedded in the matrix. In some embodiments, the RCA products can also be functionalized to form covalent attachment to the matrix preserving their spatial information within the cell thereby providing a subcellular localization distribution pattern. In some embodiments, the provided methods involve embedding RCA products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing or probe hybridization while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

### V. Detection and Analysis

In some aspects, the provided methods involve analyzing, e.g., detecting or determining, one or more nucleic acid sequences such as gap sequences in target nucleic acids. In some cases, the analysis is performed on one or more images captured, and may comprise processing the image(s) and/or quantifying signals observed. In some embodiments, the analysis comprises detecting a sequence (e.g., a gap sequence) present in the sample. In some embodiments, the analysis comprises quantification of puncta (e.g., if amplification products are detected). In some embodiments, the obtained information may be compared to a positive and negative control, or to a threshold of a feature to determine if the sample exhibits a certain feature or phenotype. In some cases, the information may comprise signals from a cell, a region, and/or comprise readouts from multiple detectable labels. In some case, the analysis further comprises displaying the information from the analysis or detection step. In some embodiments, software may be used to automate the processing, analysis, and/or display of data.

In some embodiments, one or more of the plurality of different sequences are assigned a signal code sequence, and detecting the one or more sequences comprises: i) contacting the biological sample with a first intermediate probe and a first detection oligonucleotide to generate a first complex comprising the first intermediate probe hybridized to the gap sequence in the RCP and the first detection oligonucleotide hybridized to the first intermediate probe, wherein the first intermediate probe comprises: a first hybridization region complementary to the gap sequence, and a first detectable region, and wherein the first detection oligonucleotide comprises: a sequence complementary to the first detectable region, and a first detectable label; ii) imaging the biological sample to detect a first signal from the first detectable label, wherein the first signal corresponds to a first signal code in the signal code sequence; iii) contacting the biological sample with a second intermediate probe and a second detection oligonucleotide to generate a second complex comprising the second intermediate probe hybridized to the gap sequence in the RCP and the second detection oligonucleotide hybridized to the second intermediate probe, wherein the second intermediate probe comprises: a second hybridization region complementary to the gap sequence, and a second detectable region, and wherein the second detection oligonucleotide comprises: a sequence complementary to the second detectable region, and a second detectable label; and iv) imaging the biological sample to detect a second signal from the second detectable label, wherein the second signal corresponds to a second signal code in the signal code sequence, wherein the signal code sequence comprising at least the first signal code and the second signal code is determined based on signals detected at a location in the biological sample, thereby identifying the one or more sequences of the target nucleic acid at the location in the biological sample. In some embodiments, the contacting in i) comprises contacting the biological sample with a first pool of intermediate probes and a universal pool of detection oligonucleotides, wherein the first pool of intermediate probes comprises the first intermediate probe and the universal pool of detection oligonucleotides comprises the first detection oligonucleotide and the second detection oligonucleotide, wherein each intermediate probe in the first pool of intermediate probes comprises (i) a hybridization region complementary to a gap sequence comprising one of the plurality of different sequences, and (ii) a detectable region complementary to a detection oligonucleotide of the universal pool of detection oligonucleotides, wherein the contacting in iii) comprises contacting the biological sample with a second pool of intermediate probes and the universal pool of detection oligonucleotides, wherein the second pool of intermediate probes comprises the second intermediate probe, and wherein each intermediate probe in the second pool of intermediate probes comprises (i) a hybridization region complementary to a gap sequence comprising one of the plurality of different sequences, and (ii) a detectable region complementary to a detection oligonucleotide of the universal pool of detection oligonucleotides.

In some embodiments, the first pool of intermediate probes, the second pool of intermediate probes, and one or more subsequent pools of intermediate probes are contacted with the biological sample in sequential cycles in a pre-determined sequence which corresponds to the signal code sequence assigned to the one or more sequences. In some embodiments, intermediate probes in the one or more subsequent pools each comprises (i) a hybridization region complementary to a gap sequence comprising one of the plurality of different sequences, and (ii) a detectable region complementary to a detection oligonucleotide of the universal pool of detection oligonucleotides.

In some embodiments, the method comprises identifying multiple different subsets of the plurality of different sequences of the target nucleic acid in the biological sample, wherein each subset is assigned a different signal code sequence. In some embodiments, each different subset independently comprises one or more different sequences.

In some embodiments, a first subset comprises a wildtype sequence which is assigned a first signal code sequence, and a second subset comprises two, three, four, five, or more different mutant sequences which are assigned a second signal code sequence. In some embodiments, the method comprises identifying multiple different sequences of multiple different target nucleic acids in the biological sample, wherein each subset of the different sequences of each different target nucleic acid is assigned a different signal code sequence. In some embodiments, for each different target nucleic acid a first subset of the different sequences comprises a wildtype sequence which is assigned a signal code sequence which is different from that assigned to a second subset of the different sequences. In some embodiments, the second subset comprises two, three, four, five, or more different mutant sequences.

In some embodiments, following amplification, the sequence of the amplicon (e.g., RCA product) or a portion thereof, is determined or otherwise analyzed, for example by using detectably labeled probes and imaging. The analysis of the amplification products can comprise sequencing-by-synthesis (SBS), sequencing-by-binding (SBB), avidity sequencing, sequencing-by-ligation (SBL), sequencing-by-hybridization (SBH), and/or fluorescent in situ hybridization, and/or wherein the in situ hybridization comprises sequential hybridization of probes.

In examples where SBS or SBB is used to sequence the gap sequence in the RCA product, the biological sample can be contacted with nucleotides in sequential cycles, where in each cycle a complex is formed, the complex comprising i) the sequencing primer or an extension product thereof hybridized to the sequencing primer binding site 3′ to the gap sequence, ii) a polymerase, and iii) a cognate nucleotide that base pairs with a nucleotide in the gap sequence, and a signal (ON) and/or an absence of signal (OFF) associated with the cognate nucleotide and/or the polymerase in the complex is detected at a particular location in the biological sample, wherein the ON signal, the OFF signal, or a combination thereof corresponds to the base in the cognate nucleotide and the corresponding nucleotide in the gap sequence. In some embodiments, a signal code corresponding to the ON signal, the OFF signal, or a combination thereof is detected at the particular location. In some embodiments, the signal code corresponds to a signal of a first color, a signal of a second color, a signal of a third color, or absence of signal, wherein the first, second, and third colors are different. In some embodiments, the signal code corresponds a combination of signals of a first or second color, or absence of signal, wherein the first and second colors are different. In some embodiments, the signal code corresponds to a combination of ON and/or OFF signals, wherein the combination of ON and/or OFF signals is detected in two or more imaging steps.

In some embodiments, analyzing, e.g., detecting or determining the gap sequence in the RCA product is performed using a base-by-base sequencing method, e.g., sequencing-by-synthesis (SBS), sequencing-by-avidity (SBA) or sequencing-by-binding (SBB). In some embodiments, the biological sample is contacted with a sequencing primer and base-by-base sequencing using a cyclic series of nucleotide incorporation or binding, respectively, thereby generating extension products of the sequencing primer is performed followed by removing, cleaving, or blocking the extension products of the sequencing primer.

Generally in sequencing-by-synthesis methods, a first population of detectably labeled nucleotides (e.g., dNTPs) are introduced to contact a template nucleotide (e.g., a barcode sequence in the RCP) hybridized to a sequencing primer, and a first detectably labeled nucleotide (e.g., A, T, C, or G nucleotide) is incorporated by a polymerase to extend the sequencing primer in the 5′ to 3′ direction using a complementary nucleotide (a first nucleotide residue) in the template nucleotide as template. A signal from the first detectably labeled nucleotide can then be detected. The first population of nucleotides may be continuously introduced, but in order for a second detectably labeled nucleotide to incorporate into the extended sequencing primer, nucleotides in the first population of nucleotides that have not incorporated into a sequencing primer are generally removed (e.g., by washing), and a second population of detectably labeled nucleotides are introduced into the reaction. Then, a second detectably labeled nucleotide (e.g., A, T, C, or G nucleotide) is incorporated by the same or a different polymerase to extend the already extended sequencing primer in the 5′ to 3′ direction using a complementary nucleotide (a second nucleotide residue) in the template nucleotide as template. Thus, in some embodiments, cycles of introducing and removing detectably labeled nucleotides are performed.

In some embodiments, the base-by-base sequencing comprises using a polymerase that is fluorescently labeled. In some embodiments, the base-by-base sequencing comprises using a polymerase-nucleotide conjugate comprising a fluorescently labeled polymerase linked to a nucleotide moiety that is not fluorescently labeled. In some embodiments, the base-by-base sequencing comprises using a multivalent polymer-nucleotide conjugate comprising a polymer core, multiple nucleotide moieties, and one or more fluorescent labels.

In some embodiments, the gap sequence is sequenced by SBB using a polymerase that is fluorescently labeled and one or more nucleotides that are not fluorescently labeled. In some embodiments, during SBB, a cognate nucleotide is not incorporated by the polymerase into the sequencing primer or an extension product thereof. In some embodiments, incorporation of a cognate nucleotide by the polymerase into the sequencing primer or an extension product thereof is attenuated or inhibited. Various aspects of SBB are described in U.S. Pat. No. 10,655,176 B2, the content of which is herein incorporated by reference in its entirety. In some embodiments, SBB comprises performing repetitive cycles of detecting a stabilized complex that forms at each position along the template nucleic acid to be sequenced (e.g. a ternary complex that includes the primed template nucleic acid, a polymerase, and a cognate nucleotide for the position), under conditions that prevent covalent incorporation of the cognate nucleotide into the primer, and then extending the primer to allow detection of the next position along the template nucleic acid. In the sequencing-by-binding approach, detection of the nucleotide at each position of the template occurs prior to extension of the primer to the next position. Generally, the methodology is used to distinguish the four different nucleotide types that can be present at positions along a nucleic acid template by uniquely labelling each type of ternary complex (i.e. different types of ternary complexes differing in the type of nucleotide it contains) or by separately delivering the reagents needed to form each type of ternary complex. In some instances, the labeling may comprise fluorescence labeling of, e.g., the cognate nucleotide or the polymerase that participate in the ternary complex.

In some embodiments, the gap sequence is sequenced by SBS, comprising contacting the biological sample with a nucleotide mix comprising a fluorescently labeled nucleotide and a nucleotide that is not fluorescently labeled. In some embodiments, during SBS, a cognate nucleotide is incorporated by the polymerase into the sequencing primer or an extension product thereof, and the cognate nucleotide may or may not be fluorescently labeled. In such embodiments, sequencing-by-synthesis can comprise reverse transcription and/or amplification in order to generate a template sequence from which a primer sequence can bind. Example SBS methods comprise those described for example, but not limited to, US 2007/0166705, US 2006/0188901, U.S. Pat. No. 7,057,026, US 2006/0240439, US 2006/0281109, US 2011/0059865, US 2005/0100900, U.S. Pat. No. 9,217,178, US 2009/0118128, US 2012/0270305, US 2013/0260372, and US 2013/0079232, all of which are herein incorporated by reference in their entireties.

In some embodiments, sequencing is performed by sequencing-by-avidity (SBA). Some aspects of SBA approaches are described in U.S. Pat. No. 10,768,173 B2, the content of which is herein incorporated by reference in its entirety. In some embodiments, SBA comprises detecting a multivalent binding complex formed between a fluorescently-labeled polymer-nucleotide conjugate, and a one or more primed target nucleic acid sequences (e.g., barcode sequences). Fluorescence imaging is used to detect the bound complex and thereby determine the identity of the N+1 nucleotide in the target nucleic acid sequence (where the primer extension strand is N nucleotides in length). Following the imaging step, the multivalent binding complex is disrupted and washed away, the correct blocked nucleotide is incorporated into the primer extension strand, and the sequencing cycle is repeated.

In examples where SBL is used to sequence the gap sequence in the RCA product, the biological sample can be contacted with an anchor of known sequence and detectably labeled probes, one of which are complementary to a sequence in the gap sequence in the RCA product. The anchor can be 3′ or 5′ to the gap sequence to be sequence. After hybridization of the complementary detectably labeled probe to the gap sequence, it can be ligated to the anchor or an extended product thereof, whereas detectably labeled probes that are not complementary to the gap sequence are not ligated and can be removed, e.g., by washing the sample. Signals associated with the complementary detectably labeled probe ligated to the anchor or extension product thereof can be detected, thereby detecting the corresponding sequence in the gap sequence.

In some embodiments, the detection or determination comprises hybridizing one or more probes to nucleic acid molecules such as RCPs (e.g., described in Section V). In some embodiments, the in situ detection herein can comprise sequential hybridization of probes, e.g., sequencing by hybridization and/or sequential in situ fluorescence hybridization. Sequential fluorescence hybridization can involve sequential hybridization of the intermediate probes and detectably labeled probes disclosed herein. In some embodiments, a method disclosed herein comprises sequential hybridization of detectably labeled probes and intermediate probes that are not detectably labeled per se but are capable of binding (e.g., via nucleic acid hybridization) and being detected by detectably labeled probes, such as fluorescently labeled probes. Exemplary methods comprising sequential fluorescence hybridization of probes are described in US 2019/0161796, US 2020/0224244, US 2022/0010358, US 2021/0340618, WO 2020/123742, and WO 2021/138676, all of which are incorporated herein by reference.

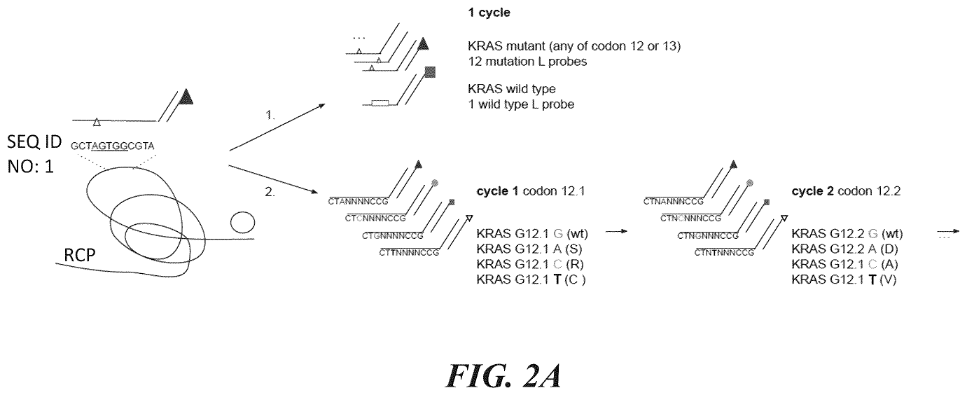

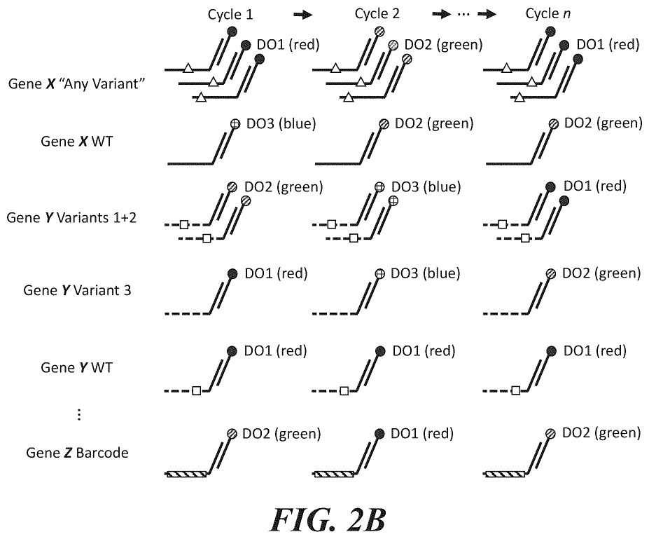

In some embodiments, the detection or determination comprises detecting a gap sequence in a temporally sequential manner for in situ analysis in a biological sample, e.g., in an intact tissue. In some aspects, provided herein is a method for detecting the detectably labeled probes, thereby generating a signal code. In some instances, each signal code corresponds to a variant sequence comprised by a gap sequence. In some instances, one signal code corresponds to a wildtype sequence comprised by a gap sequence while a different signal code corresponds to any mutated sequence compared to the wildtype sequence (e.g., a mutant sequence). In some instances, the probes may be optically detected (e.g., by detectably labeled probes) in a temporally-sequential manner. In some embodiments, the sample is contacted with a library of probes to detect the probes or products thereof (e.g., used or generated as described in Section III) associated with the gap sequence in each target nucleic acid. For example, each gap sequence is assigned a different signal code sequence and corresponds to a splint oligonucleotide of a library of splint oligonucleotides (e.g., described in Section III). In some instances, the probes or probe sets comprising various probe types may be applied to a sample simultaneously. In some instances, the probes or probe sets comprising various probe types may be applied to a sample sequentially. In some aspects, the method comprises sequential hybridization of labelled probes to create a signal code sequence (e.g., a temporal pattern of signal codes corresponding to signals detected at a location) that identifies the gap sequence or portion thereof. In some instances, each splint oligonucleotide from a library (e.g., described in Section III) is associated with a different signal code sequence. In some instances, the splint oligonucleotide corresponding to the wildtype sequence is associated with a signal code sequence, and the splint oligonucleotides corresponding to all mutated sequences are associated with a different signal code sequence that is shared among the mutated sequences. In such instances, the readout is binary with the second signal code sequence at a location in the sample indicating the presence of any mutation in the gap sequence of target nucleic acid molecules at the location, and the absence of the second signal code sequence and presence of the first signal code sequence indicating the absence of any mutation in the gap sequence of target nucleic acid molecules at the location. For example, as shown in FIG. 2A, upper panel, a one-cycle probe hybridization and detection can be used to detect whether any one or more particular sequence variants (e.g., mutations) are present or not at location(s) in a biological sample. Alternatively, multiple cycles of probe hybridization and detection can be performed to detect multiple sequence variants (e.g., more than three, four, five, six, seven, eight, or more, including wildtype sequence) at location(s) in a biological sample, as shown in FIG. 2A, lower panel.

In some embodiments, provided herein is a method for analyzing a biological sample, comprising: a) contacting the biological sample with a plurality of circularizable probes, wherein each circularizable probe comprises a first probe region and a second probe region that hybridize to a first target sequence and a second target sequence, respectively, in a target RNA in the biological sample, wherein the first and second target sequences are separated by a gap sequence in the target RNA, wherein the gap sequence in a wildtype molecule of the target RNA comprises a wildtype sequence, and wherein the gap sequence in a mutant molecule of the target RNA comprises a mutant sequence; b) contacting the biological sample with a library of splint oligonucleotides that compete for hybridization to the gap sequence, the library of splint oligonucleotides comprising: i) a wildtype splint oligonucleotide comprising ligatable ends and a hybridization region complementary to the gap sequence comprising the wildtype sequence of the target RNA, ii) a mutant splint oligonucleotide comprising ligatable ends and a hybridization region complementary to the gap sequence comprising the mutant sequence of the target RNA; c) ligating a splint oligonucleotide to a circularizable probe hybridized to a molecule of the target RNA, thereby circularizing the circularizable probe to generate a circularized probe comprising a gap-filled region complementary to the gap sequence in the target RNA; d) generating a rolling circle amplification product (RCP) of the circularized probe in the biological sample, wherein the RCP comprises multiple copies of the gap sequence; e) contacting the biological sample with a plurality of intermediate probes comprising: i) a wildtype intermediate probe comprising a wildtype hybridization region complementary to the gap sequence comprising the wildtype sequence of the target RNA, wherein the wildtype intermediate probe hybridizes to a detection oligonucleotide comprising a detectable label of a first color; and ii) a mutant intermediate probe comprising a mutant hybridization region complementary to the gap sequence comprising the mutant sequence of the target RNA, wherein the mutant intermediate probe hybridizes to a detection oligonucleotide comprising a detectable label of a second color, wherein the intermediate probe or the mutant probe hybridizes to the gap sequence in the RCP; and detecting a signal associated with the detectable label of the first color or the second color at a location in the biological sample, thereby identifying the wildtype sequence or the mutant sequence of the target RNA at the location in the biological sample. In some embodiments, the plurality of circularizable probes hybridize to both the wildtype molecule and the mutant molecule of the target RNA. In some embodiments, the biological sample comprises different molecules of the target RNA each comprising a mutant sequence of a plurality of different mutant sequences. In some embodiments, the library of splint oligonucleotides comprises different mutant splint oligonucleotides each comprising a hybridization region complementary to a mutant sequence of a plurality of different mutant sequences. In some embodiments, the plurality of intermediate probes comprises different mutant intermediate probes each comprising a mutant sequence of a plurality of different mutant sequences. In some embodiments, the biological sample is a formalin-fixed, paraffin-embedded (FFPE) sample, a frozen tissue sample, or a fresh tissue sample.

In some embodiments, provided herein is a method of analyzing a sample, comprising: a) producing an RCA product in the sample, the RCA product comprising multiple copies of a gap sequence, wherein the gap sequence corresponds to a splint oligonucleotide sequence and is assigned a signal code, and wherein the sample is a cell or tissue sample; b) contacting the sample with a first intermediate probe and a first detectably labeled probe to generate a first complex comprising the first intermediate probe hybridized to the RCA product and the first detectably labeled probe (e.g., a detection oligonucleotide (DO), such as any one of DO1, DO2, and DO3 in FIG. 2B) hybridized to the first intermediate probe, wherein the first intermediate probe comprises (i) a hybridization region complementary to the gap sequence or a portion thereof and (ii) a first overhang sequence, and wherein the first detectably labeled probe comprises (i) a sequence complementary to the first overhang sequence and (ii) a first optically detectable moiety; c) imaging the sample to detect a first signal from the first optically detectable moiety, wherein the first signal corresponds to a first signal code in the signal code sequence; d) contacting the sample with a second intermediate probe and a second detectably labeled probe (e.g., a DO such as any one of DO1, DO2, and DO3 in FIG. 2B) to generate a second complex comprising the second intermediate probe hybridized to the RCA product and the second detectably labeled probe hybridized to the second intermediate probe, wherein the second intermediate probe comprises (i) a hybridization region complementary to the gap sequence and (ii) a second overhang sequence, and wherein the second detectably labeled probe comprises (i) a sequence complementary to the second overhang sequence and (ii) a second optically detectable moiety; and e) imaging the sample to detect a second signal from the second optically detectable moiety, wherein the second signal corresponds to a second signal code in the signal code sequence, wherein the signal code sequence comprising at least the first signal code and the second signal code is determined at a location in the sample, thereby decoding and identifying the gap sequence at the location in the sample.

In some embodiments, a detectably labeled probe can hybridize to a detectable region in an intermediate probe. In some embodiments, the detectable region is in a 5′ overhang and/or a 3′ overhang of the intermediate probe, upon hybridization of the intermediate probe to the gap sequence or a portion thereof in an RCP. In some embodiments, the detectable region is a split region, e.g., a portion of the detectable region can be in the 5′ overhang and another portion of the detectable region can be in the 3′ overhang of an intermediate probe. In some embodiments, the detectable region is in the 5′ overhang of the intermediate probe. In some embodiments, the detectable region is in the 3′ overhang of the intermediate probe. In some embodiments, a first portion of the detectable region is in the 3′ overhang and a second portion of the detectable region is in the 5′ overhang of the intermediate probe.

As shown in FIG. 1, a library of splint oligonucleotides and a circularizable probe (e.g., a common circularizable probe for multiple different mutations to be detected) are designed for detection of mutations in a gap sequence in a target nucleic acid in a biological sample. The library of splint oligonucleotides comprises splint oligonucleotide molecules that are complementary to the wildtype sequence of the gap sequence and splint oligonucleotides molecules that are complementary to wildtype or mutated sequences (e.g., point mutations) in the gap sequence. The common circularizable probe is designed to hybridize to conserved regions outside of the gap sequence. Upon contacting the biological sample, the library of splint oligonucleotides compete with one another to hybridize to the target nucleic acid at the gap sequence and ligate to the circularizable probe to form a circularized probe. The circularized probes are amplified via RCA to generate RCPs that each comprises multiple copies of the gap sequence. Detection of the target sequences can be performed using intermediate probes each designed to hybridize to at least a portion of a copy of a gap sequence in an RCP. The readout signals can be detected by hybridizing detectably labeled probes to the intermediate probes. In some embodiments, readout signals can be detected by hybridizing detectably labeled probes directly to a sequence of the RCA product (e.g., at least a portion of a copy of a gap sequence in an RCP).

In some embodiments, the target nucleic acid is among a plurality of target nucleic acids, wherein the method comprises contacting the sample with a first pool of intermediate probes and a universal pool of detectably labeled probes, wherein the first pool of intermediate probes comprises the first intermediate probe and the universal pool of detectably labeled probes comprises the first detectably labeled probe and the second detectably labeled probe, wherein each intermediate probe in the first pool of intermediate probes comprises (i) a hybridization region complementary to one of the plurality of target nucleic acids and (ii) an overhang sequence complementary to a detectably labeled probe of the universal pool of detectably labeled probes; and the method comprises contacting the sample with a second pool of intermediate probes and the universal pool of detectably labeled probes, wherein the second pool of intermediate probes comprises the second intermediate probe, and wherein each intermediate probe in the second pool of intermediate probes comprises (i) a hybridization region complementary to one of the plurality of target nucleic acid and (ii) an overhang sequence complementary to a detectably labeled probe of the universal pool of detectably labeled probes. In some embodiments, the first and second pool of intermediate probes hybridize to at least part of the common regions adjacent to the gap sequence in the RCA product. In some embodiments, the first and the second pool of intermediate probes are contacted with the biological sample simultaneously.

In some embodiments, the method comprises identifying multiple different gap sequences present at locations in the sample, wherein each different gap sequence is assigned a different signal code sequence and is targeted by a circularizable probe or probe set and a splint oligonucleotide comprising a complement of a different target nucleic acid of the plurality of target nucleic acid. In some embodiments, the number of different intermediate probes in each pool of intermediate probes is greater than the number of different detectably labeled probes in the universal pool of detectably labeled probes. In some embodiments, the number of different detectably labeled probes in the universal pool of reporter probes is two, three, four, five, six, or more. In some embodiments, the number of different intermediate probes in each pool of intermediate probes is about 4, about 10, about 20, about 50, about 100, about 200, about 500, about 1,000, about 2,000, about 5,000, or more.

In some embodiments, the different intermediate probes in a pool of intermediate probes correspond to a library of splint oligonucleotides, e.g., as described in Section III-B. In some embodiments, the hybridization sequence of an intermediate probe is complementary to the gap sequence in an RCA product and is the same as the corresponding gap sequence in a target nucleic acid which is used as a template to circularize the circularizable probe for the RCA. In some embodiments, the hybridization sequence of an intermediate probe comprises a sequence that is the same as the splint oligonucleotide sequence used to fill the gap of the circularizable probe hybridized to the target nucleic acid. In some embodiments, the hybridization sequence of an intermediate probe comprises a sequence that is the same as the gap sequence in the target nucleic acid. In some embodiments, the hybridization sequence of an intermediate probe comprises a sequence that is the same as at least part of the common regions adjacent to the gap sequence in the target nucleic acid.

Provided herein are methods involving the use of one or more probes for analyzing one or more target nucleic acid(s), such as variant sequences in one or more target nucleic acids present in a cell or a biological sample, such as a tissue sample. In some embodiments, the probes can include a plurality of intermediate probes and a plurality of detectably labeled probes for combinatorially decoding the hybridization regions of the intermediate probes; since the hybridization regions of the intermediate probes can comprise identifier sequences (e.g., target analyte sequences or barcode sequences), the target nucleic acid in the sample can be identified by decoding the identifier sequences using sequential probe hybridization. In some aspects, the provided embodiments can be employed for in situ detection of variant sequences in target nucleic acids in a cell, e.g., in cells of a biological sample or a sample derived from a biological sample, such as a tissue section on a solid support, such as on a transparent slide.

For example, to detect mutations in different gene transcripts, a pool of intermediate probes can contain two intermediate probes for each gene, one for the wildtype and the other for “any variant” of the gene. For example, as shown in FIG. 2B, in the pool of intermediate probes for a particular cycle, the two intermediate probes for the same gene X can carry different overhangs, each different overhang designed to hybridize to a detectably labeled probe with a different color (e.g., Cycle 1 where Gene X WT corresponds to DO3 (blue), whereas Gene X “Any Variant” corresponds to DO1 (red)). In the pool of intermediate probes for another particular cycle, the two intermediate probes for gene X can carry the same overhang designed to hybridize to the same detectably labeled probe (e.g., Cycle 1 where Gene X WT and Gene X “Any Variant” both correspond to DO2 (green)), as long as the signal code sequences for gene X (wildtype) and gene X (“any variant”) detected through the sequential probe hybridization cycles can distinguish gene X (“any variant”) from gene X (wildtype), and from gene Y (wildtype), gene Y (“Variants 1+2”), and gene Y (“Variant 3”). In the particular example, a codebook can be generated comprising a first code for gene X (wildtype) and a second code for gene X (“any variant”), as well as a third code for gene Y (wildtype), a fourth code for gene Y (“Variants 1+2”), and fifth code for gene Y (“Variant 3”), where the first, second, third, fourth, and fifth codes can be distinct codes. In some examples, a codebook can comprise more than two codes for gene X, for instance, a first code for wildtype X, a second code for variant X1, and a third code for variant X2, etc. In some examples, a codebook can comprise only one code for gene Z, for instance, where detection of gene Z transcripts in desirable but identification of any particular one or more gene Z sequence variants or mutants is not required. In some examples, a codebook can comprise any combinations of the aforementioned codes, for instance, a code for gene X (wildtype), a code for gene X (“any variant”), a code for variant X1, a code for gene Y (wildtype), a code for variant Y1, a code for variant Y2, and/or a code for gene Z, and the corresponding intermediate probes and detectably labeled probes can be designed to detect the genes and/or sequence variants thereof in a sample. The codebook and readout schemes can be used to identify sequence variants in RCPs generated using circularized probe gap-filled by polymerization, splint oligonucleotide ligation, or a combination thereof.

In the example shown in FIG. 2B, a universal pool of detection oligonucleotides (DOs) DO1 (red, “R”), DO2 (green, “G”), and DO3 (blue, “B”) are used to detect the library of intermediate probes used in sequential cycles Cycle 1, Cycle 2, . . . , Cycle n. Using the exemplary coding scheme, the codebook can comprise the following signal code sequences:
RGR for Gene X (“Any Variant”);
BGG for Gene X (WT);
GBR for Gene Y (Variant 1+Variant 2);
RBG for Gene Y (Variant 3);
RRR for Gene Y (WT); and
GRG for Gene Z (Barcode).

In some aspects, provided herein are in situ assays using microscopy as a readout, e.g., hybridization, or other detection or determination methods involving an optical readout. In some aspects, detection or determination of a sequence of one, two, three, four, five, or more nucleotides of a gap sequence in a target nucleic acid is performed in situ in a cell in an intact tissue. In some aspects, detection or determination of a sequence is performed such that the localization of the target nucleic acid (or product or a derivative thereof associated with the target nucleic acid) in the originating sample is detected. In some embodiments, the assay comprises detecting the presence or absence of an amplification product or a portion thereof (e.g., RCA product or hybridization complex). In some embodiments, a provided method is quantitative and preserves the spatial information within a tissue sample without physically isolating cells or using homogenates. In some embodiments, the present disclosure provides methods for high-throughput profiling of target nucleic acids in situ in a large number of cells, tissues, organs or organisms.

In some aspects, the provided methods comprise imaging the amplification product (e.g., RCA product) via binding of an intermediate probe and a detectably labeled probe (e.g., a detection oligonucleotide comprising a fluorescent label), and detecting the detectable label. In some embodiments, a signal associated with a detectably labeled oligonucleotide can be measured and quantitated. The terms “label” and “detectable label” comprise a directly or indirectly detectable moiety that is associated with (e.g., conjugated to) a molecule to be detected, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

The term “fluorophore” comprises a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (Max Vision Biosciences), and/or a very intense black dye (e.g., Sudan Black, or comparable dark chromophore).

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. Nos. 4,757,141, 5,151,507 and 5,091,519. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264. As used herein, the term “fluorescent label” comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

In some embodiments, an RCP or a probe disclosed herein comprises one or more detectably labelled, e.g., fluorescent, nucleotides. In some embodiments, the one or more detectably labelled nucleotides are incorporated during generation of the RCP (e.g., during RCA) or the probe. Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). For exemplary methods for custom synthesis of nucleotides having other fluorophores, see, Henegariu et al. (2000) Nature Biotechnol. 18:345.

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. In some embodiments, the term antibody refers to an antibody molecule of any class, or any sub-fragment thereof, such as a Fab.

Other suitable labels for a polynucleotide sequence may comprise fluorescein (FAM), digoxigenin, dinitrophenol (DNP), dansyl, biotin, bromodeoxyuridine (BrdU), hexahistidine (6xHis), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or an polynucleotide sequence can be indirectly labeled, especially with a hapten that is then bound by a capture agent, e.g., as disclosed in U.S. Pat. Nos. 5,344,757, 5,702,888, 5,354,657, 5,198,537 and 4,849,336, and PCT publication WO 91/17160. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of an RCP, an intermediate probe, and/or a detectably labeled oligonucleotide bound to the RCP or to the intermediate probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of an RCP, an intermediate probe, and/or a detectably labeled oligonucleotide bound to the RCP or to the intermediate probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (i.e., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECS™), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PS™, photon scanning tunneling microscopy (PS™), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXS™), and intact tissue expansion microscopy (exM).

### VI. Samples and Sample Processing

A sample disclosed herein can be derived from any biological sample. The sample may not be limited to any specific source, but may be peripheral blood mononuclear cells, tumors, tissue, bone marrow, biopsies, serum, blood, plasma, saliva, lymph fluid, pleura fluid, cerebrospinal and synovial fluid. The sample may be extracted from a subject. Samples extracted from individuals may be subjected to the methods described herein to identify and evaluate immune responses during cancer and disease or subsequent to immunotherapy.

Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components.

Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

In some embodiments, the biological sample corresponds to cells (e.g., derived from a cell culture, a tissue sample, or cells deposited on a surface). In a cell sample with a plurality of cells, individual cells can be naturally unaggregated. For example, the cells can be derived from a suspension of cells (e.g., a body fluid such as blood) and/or disassociated or disaggregated cells from a tissue or tissue section. The number of cells in the biological sample can vary. Some biological samples comprise large numbers of cells, e.g., blood samples, while other biological samples comprise smaller or only a small number of cells or may only be suspected of containing cells, e.g., plasma, serum, urine, saliva, synovial fluids, amniotic fluid, lachrymal fluid, lymphatic fluid, liquor, cerebrospinal fluid and the like.

In some embodiments, a cell-containing biological sample can comprise a body fluid or a cell-containing sample derived from the body fluid, e.g., whole blood, samples derived from blood such as plasma or serum, buffy coat, urine, sputum, lachrymal fluid, lymphatic fluid, sweat, liquor, cerebrospinal fluid, ascites, milk, stool, bronchial lavage, saliva, amniotic fluid, nasal secretions, vaginal secretions, semen/seminal fluid, wound secretions, cell culture and swab samples, or any cell-containing sample derived from the aforementioned samples. In some embodiments, a cell-containing biological sample can be a body fluid, a body secretion or body excretion, e.g., lymphatic fluid, blood, buffy coat, plasma or serum. In some embodiments, a cell-containing biological sample can be a circulating body fluid such as blood or lymphatic fluid, e.g., peripheral blood obtained from a mammal such as human.

The biological sample can include any number of macromolecules, for example, cellular macromolecules and organelles (e.g., mitochondria and nuclei). The biological sample can be obtained as a tissue sample, such as a tissue section, biopsy, a core biopsy, needle aspirate, or fine needle aspirate. In some embodiments, the biological sample is or comprise a cell pellet or a section of a cell pellet. In some embodiments, the biological sample is or comprise a cell block or a section of a cell block. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface. In some embodiments, the biological sample may comprises transcripts of antigen receptor molecules.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or padlock probe. In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a padlock probe.

In some embodiments, one or more post-fixing step is performed after contacting a sample with a binding or labelling agent (e.g., an antibody or antigen binding fragment thereof) for a non-nucleic acid analyte such as a protein analyte. The labelling agent can comprise a nucleic acid molecule (e.g., reporter oligonucleotide) comprising a sequence corresponding to the labelling agent and therefore corresponds to (e.g., uniquely identifies) the analyte. In some embodiments, the labelling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221): 543-548, 2015, the entire contents of which are incorporated herein by reference.

### (v) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Methods of any suitable destaining or discoloring a biological sample can be used, and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) can be isometrically expanded. Isometric expansion methods that can be used include hydration, a preparative step in expansion microscopy, as described in Chen et al., Science 347(6221):543-548, 2015.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, MA), Label-IT Amine (available from MirusBio, Madison, WI) and Label X (described for example in Chen et al., Nat. Methods 13:679-684, 2016, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-Crosslinking

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g., PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labelling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked.

(viii) Tissue Permeabilization and Treatment

In some embodiments, a biological sample can be permeabilized to facilitate transfer of species (such as probes) into the sample. If a sample is not permeabilized sufficiently, the amount of species (such as probes) in the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. Non-chemical permeabilization methods can be used. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

(ix) Selective Enrichment of RNA or cDNA Species

In some embodiments, where RNA or cDNA is the analyte, one or more RNA or cDNA analyte species of interest can be selectively enriched. For example, one or more species of RNA or cDNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs or cDNAs of interest can be used to amplify the one or more RNAs or cDNAs of interest, thereby selectively enriching these RNAs or cDNAs.

In some aspects, when two or more analytes are analyzed, a first and second probe that is specific for (e.g., specifically hybridizes to) each RNA or cDNA analyte are used. For example, in some embodiments of the methods provided herein, templated ligation is used to detect gene expression in a biological sample. An analyte of interest (such as a protein), bound by a labelling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include amplification of templated ligation products (e.g., by multiplex PCR).

In some embodiments, an oligonucleotide with sequence complementarity to the complementary strand of captured RNA (e.g., cDNA) can bind to the cDNA. For example, biotinylated oligonucleotides with sequence complementary to one or more cDNA of interest binds to the cDNA and can be selected using biotinylation-strepavidin affinity using any of a variety of suitable methods (e.g., streptavidin beads).

In some embodiments, the analytes may be further enriched for in situ readout by immobilization at a location in the biological sample. In a non-limiting example, the analytes may comprise one or more fragments that are specific to a location in the biological sample.

Alternatively, one or more species of RNA can be down-selected (e.g., removed) using any of a variety of methods. For example, probes can be administered to a sample that selectively hybridize to ribosomal RNA (rRNA), thereby reducing the pool and concentration of rRNA in the sample. Additionally and alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the entire contents of which are incorporated herein by reference). Furthermore, hydroxyapatite chromatography can remove abundant species (e.g., rRNA) (see, e.g., Vandernoot, V.A., cDNA normalization by hydroxyapatite chromatography to enrich transcriptome diversity in RNA-seq applications, Biotechniques, 53(6) 373-80, (2012), the entire contents of which are incorporated herein by reference).

A biological sample may comprise one or a plurality of analytes of interest. Methods for performing multiplexed assays to analyze two or more different analytes in a single biological sample are provided.

### VII. Compositions, Kits, and Systems

Provided herein are kits, for example comprising one or more oligonucleotides, e.g., any described in Sections I-V, and instructions for performing the methods provided herein. In some embodiments, the kits further comprise one or more reagents for performing the methods provided herein. In some embodiments, the kits further comprise one or more reagents required for one or more steps comprising hybridization, ligation, extension, amplification, detection, and/or sample preparation as described herein. In some embodiments, the kit further comprises any one or more of the intermediate probes and detectably labeled oligonucleotides disclosed herein, e.g., as described in Sections V. In some embodiments, any or all of the oligonucleotides are DNA molecules. In some embodiments, the kit further comprises an enzyme such as a ligase and/or a polymerase described herein. In some embodiments, the ligase has DNA-splinted DNA ligase activity. In some embodiments, the ligase has DNA-templated ligase activity. In some embodiments, the kit comprises a polymerase, for instance for performing extension of the primers to incorporate modified nucleotides into cDNA products of antigen receptor transcripts. In some embodiments, the kits may contain reagents for forming a functionalized matrix (e.g., a hydrogel), such as any suitable functional moieties. In some examples, also provided are buffers and reagents for tethering the modified primers, cDNA products, and/or RCA products to the functionalized matrix. The various components of the kit may be present in separate containers or certain compatible components may be pre-combined into a single container. In some embodiments, the kits further contain instructions for using the components of the kit to practice the provided methods.

In some embodiments, provided herein is a kit for analyzing a biological sample, comprising: a) a circularizable probe comprising a first probe region and a second probe region that hybridize to a first target sequence and a second target sequence, respectively, in a target nucleic acid (e.g., RNA) in the biological sample, wherein the first and second target sequences are separated by a gap sequence in the target nucleic acid (e.g., RNA), and the gap sequence comprises a variant sequence among a plurality of different variant sequences; b) one or more reagents for circularizing the circularizable probe to generate a circularized probe comprising a gap-filled region complementary to the gap sequence; and/or c) one or more reagents for generating a rolling circle amplification product (RCP) of the circularized probe, wherein the RCP comprises multiple copies of the gap sequence.

In some embodiments, a kit disclosed herein comprises a plurality of intermediate probes, wherein each intermediate probe comprises: i) a hybridization region complementary to one of the plurality of different variant sequences; and ii) a detectable region that hybridizes to a detection oligonucleotide comprising a detectable label corresponding to the hybridization region in the intermediate probe. In some embodiments, the detectable label can correspond to one or more of the plurality of different variant sequences. For example, a detectable label of a first color (e.g., a green fluorescent label) can correspond to one variant sequence (e.g., a wildtype nucleotide in KRAS), and a detectable label of a second color (e.g., a red fluorescent label) can correspond to any one, two, three, or more other variant sequences (e.g., mutant nucleotides in KRAS). In some embodiments, an intermediate probe of the plurality of intermediate probes hybridizes to one of the multiple copies of the gap sequence in the RCP.

In some embodiments, a kit disclosed herein comprises a pool of detection oligonucleotides each comprising a detectable label. In some embodiments, the biological sample is imaged to detect signals associated with the detectable labels at locations in the biological sample, thereby detecting one or more of the plurality of different variant sequences in the biological sample. In some embodiments, the one or more of the plurality of different variant sequences are identified (e.g., the identity of an SNP or point mutation is revealed) in the biological sample, based on the signals detected at the locations.

In some embodiments, a kit disclosed herein comprises a) a circularizable probe comprising a first probe region and a second probe region that hybridize to a first target sequence and a second target sequence, respectively, in a target nucleic acid in the biological sample, wherein the first and second target sequences are separated by a gap sequence in the target nucleic acid, and wherein the gap sequence comprises a variant sequence among a plurality of different variant sequences; and b) a library of splint oligonucleotides, wherein each splint oligonucleotide comprises: i) ligatable ends; and ii) a hybridization region complementary to one of the plurality of different variant sequences, wherein a splint oligonucleotide of the library of splint oligonucleotides that is complementary to the gap sequence is ligated to the circularizable probe, thereby circularizing the circularizable probe to generate a circularized probe comprising a gap-filled region complementary to the gap sequence. In some embodiments, each splint oligonucleotide of the library comprises a phosphate group on the 5′-end available for ligation.

The biological sample, the circularizable probe (e.g., in a composition comprising a plurality of circularizable probes), and the library of splint oligonucleotides can be contacted with one another in any order. For instance, the circularizable probe and the library of splint oligonucleotides can be pre-mixed prior to contacting the biological sample with the mixture. In other examples, the biological sample can be contacted with the circularizable probe and then with the library of splint oligonucleotides. In yet other examples, the biological sample can be contacted with the library of splint oligonucleotides and then with the circularizable probe. In still other examples, the circularizable probe and the library of splint oligonucleotides are provided in separate compositions which are contacted with the biological sample simultaneously. In some embodiments, the method comprises generating a rolling circle amplification product (RCP) of the circularized probe in the biological sample, wherein the RCP comprises multiple copies of the gap sequence. In some embodiments, the method comprises detecting a sequence comprising the variant sequence in the gap sequence of the RCP at a location in the biological sample, thereby detecting the target nucleic acid comprising the variant sequence at the location in the biological sample. The method may but does not need to comprise detecting a barcode sequence in the RCP.

In some embodiments, a kit disclosed herein comprises a plurality of intermediate probes (e.g., nucleic acid probes), wherein each intermediate probe comprises: i) a hybridization region complementary to one of the plurality of different variant sequences; and ii) a detectable region that hybridizes to a detection oligonucleotide comprising a detectable label, wherein an intermediate probe of the plurality of intermediate probes hybridizes to one of the multiple copies of the gap sequence in the RCP. In some embodiments, the method comprises detecting signals associated with the detectable labels at locations in the biological sample, thereby identifying one or more of the plurality of different variant sequences in the biological sample.

In some embodiments, a kit disclosed herein comprises a circularizable probe comprising a first probe region and a second probe region that hybridize to a first target sequence and a second target sequence, respectively, in a target RNA in the biological sample, wherein the first and second target sequences are separated by a gap sequence in the target RNA, and wherein the gap sequence comprises a variant sequence among a plurality of different variant sequences. In some embodiments, a kit disclosed herein comprises reagents for circularizing the circularizable probe to generate a circularized probe comprising a gap-filled region complementary to the gap sequence. In some embodiments, a kit disclosed herein comprises reagents for generating a rolling circle amplification product (RCP) of the circularized probe in the biological sample, wherein the RCP comprises multiple copies of the gap sequence. In some embodiments, a kit disclosed herein comprises reagents for detecting a sequence comprising the variant sequence in the gap sequence of the RCP at a location in the biological sample, thereby detecting the target RNA comprising the variant sequence at the location in the biological sample. In some embodiments, a kit disclosed herein comprises reagents for base-by-base sequencing of the sequence comprising the variant sequence, and the base-by-base sequencing may comprise determining the identity of one, two, three, or more bases per cycle in sequential sequencing cycles. In some embodiments, a kit disclosed herein comprises a plurality of intermediate probes (e.g., nucleic acid probes), wherein each intermediate probe comprises: i) a hybridization region complementary to one of the plurality of different variant sequences; and ii) a detectable region that hybridizes to a detection oligonucleotide comprising a detectable label, wherein an intermediate probe of the plurality of intermediate probes hybridizes to the multiple copies of the gap sequence in the RCP. In some embodiments, a kit disclosed herein comprises reagents for detecting signals associated with the intermediate probes of the plurality of intermediate probes in sequential probe hybridization cycles.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as detectably labeled oligonucleotides or detectable labels. In some embodiments, the kits optionally contain other components, for example nucleic acid primers, enzymes and reagents, buffers, nucleotides, modified nucleotides, reagents for additional assays.

### VIII. Applications

In some aspects, the provided embodiments can be applied in an in situ method of analyzing nucleic acid sequences in intact tissues or samples in which the spatial information has been preserved. In some aspects, the embodiments can be applied in an imaging or detection method for multiplexed nucleic acid analysis. In some aspects, the provided embodiments can be used to identify or detect mutations in a target nucleic acid. In some aspects, the provided embodiments can be used to crosslink the RCA products via modified nucleotides, e.g., to a matrix, to increase the stability of the circularizable probe or probe set, or the RCA products in situ.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples. In some aspects, the embodiments can be applied to visualize the distribution of genetically encoded markers in whole tissue at subcellular resolution.

### IX. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” “polynucleotide,” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

In some embodiments, “hybridization” as used herein refers to the process in which two single-stranded polynucleotides bind non-covalently to form a stable double-stranded polynucleotide. In one aspect, the resulting double-stranded polynucleotide can be a “hybrid” or “duplex.” “Hybridization conditions” typically include salt concentrations of approximately less than 1 M, often less than about 500 mM and may be less than about 200 mM. A “hybridization buffer” includes a buffered salt solution such as 5% SSPE, or other such buffers. Hybridization temperatures can be as low as 5° C., but are typically greater than 22° C., and more typically greater than about 30° C., and typically in excess of 37° C. Hybridizations are often performed under stringent conditions, e.g., conditions under which a sequence will hybridize to its target sequence but will not hybridize to other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the Tₘ for the specific sequence at a defined ionic strength and pH. The melting temperature Tₘ can be the temperature at which a population of double-stranded nucleic acid molecules becomes half dissociated into single strands. Several equations for calculating the Tₘ of nucleic acids can be used. As indicated by standard references, a simple estimate of the Tₘ value may be calculated by the equation, Tₘ=81.5+0.41 (% G+C), when a nucleic acid is in aqueous solution at 1 M NaCl (see e.g., Anderson and Young, Quantitative Filter Hybridization, in Nucleic Acid Hybridization (1985)). Other references (e.g., Allawi and SantaLucia, Jr., Biochemistry, 36:10581-94 (1997)) include alternative methods of computation which take structural and environmental, as well as sequence characteristics into account for the calculation of Tₘ.

In general, the stability of a hybrid is a function of the ion concentration and temperature. Typically, a hybridization reaction is performed under conditions of lower stringency, followed by washes of varying, but higher, stringency. Exemplary stringent conditions include a salt concentration of at least 0.01 M to no more than 1 M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized. In one aspect, “stringency of hybridization” in determining percentage mismatch can be as follows: 1) high stringency: 0.1×SSPE, 0.1% SDS, 65° ° C.; 2) medium stringency: 0.2×SSPE, 0.1% SDS, 50° C. (also referred to as moderate stringency); and 3) low stringency: 1.0×SSPE, 0.1% SDS, 50° ° C. It is understood that equivalent stringencies may be achieved using alternative buffers, salts and temperatures. For example, moderately stringent hybridization can refer to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions can be conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. Low stringency hybridization can refer to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M EDTA. Other suitable moderate stringency and high stringency hybridization buffers and conditions are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See M. Kanehisa, Nucleic Acids Res. 12:203 (1984).

A “primer” used herein is, in some embodiments, an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

In some embodiments, “ligation” refers to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

“Sequencing,” “sequence determination” and the like means determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid. “High throughput digital sequencing” or “next generation sequencing” means sequence determination using methods that determine many (typically thousands to billions) of nucleic acid sequences in an intrinsically parallel manner, i.e. where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technologies, Inc., Carlsbad, Calif.); sequencing by synthesis using modified nucleotides (such as commercialized in TruSeq™ and HiSeq™ technology by Illumina, Inc., San Diego, Calif.; HeliScope™ by Helicos Biosciences Corporation, Cambridge, Ma.; and PacBio RS by Pacific Biosciences of California, Inc., Menlo Park, Calif.), sequencing by ion detection technologies (such as Ion Torrent™ technology, Life Technologies, Carlsbad, Calif.); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods. “Multiplexing” or “multiplex assay” herein may refer to an assay or other analytical method in which the presence and/or amount of multiple targets, e.g., multiple nucleic acid target sequences, can be assayed simultaneously by using more than one probes, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout the present disclosure, various aspects are presented in a range format. It should be understood that the description in range format is merely for convenience and brevity and should not be construed as an inflexible limitation on the scope of the present disclosure. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the present disclosure. The upper and lower limits of these smaller ranges may independently be comprised in the smaller ranges, and are also encompassed within the present disclosure, subject to any specifically excluded limit in the stated range. Where the stated range comprises one or both of the limits, ranges excluding either or both of those comprised limits are also comprised in the present disclosure. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### Exemplary Embodiments

1. A method for analyzing a biological sample, comprising:
a) contacting the biological sample with a circularizable probe,
wherein the circularizable probe comprises a first probe region and a second probe region that hybridize to a first target sequence and a second target sequence, respectively, in a target nucleic acid in the biological sample,
wherein the first and second target sequences are separated by a gap sequence in the target nucleic acid, and
wherein the gap sequence comprises a variant sequence among a plurality of different sequences;
b) circularizing the circularizable probe to generate a circularized probe comprising a gap-filled region complementary to the gap sequence;
c) generating a rolling circle amplification product (RCP) of the circularized probe in the biological sample, wherein the RCP comprises multiple copies of the gap sequence;
d) contacting the biological sample with a plurality of nucleic acid probes, wherein each nucleic acid probe:
   i) comprises a hybridization region complementary to one of the plurality of different sequences; and
   ii) is detectably labeled or comprises a detectable region that directly or indirectly binds to a detection oligonucleotide comprising a detectable label, wherein a nucleic acid probe of the plurality of nucleic acid probes hybridizes to one of the multiple copies of the gap sequence in the RCP; and
e) detecting a signal associated with the nucleic acid probe at a location in the biological sample, thereby identifying the variant sequence in the biological sample.

2. The method of embodiment 1, wherein circularizing the circularizable probe comprises contacting the biological sample with a library of splint oligonucleotides, wherein each splint oligonucleotide comprises:
i) ligatable ends; and
ii) a hybridization region complementary to one of the plurality of different sequences,
wherein a splint oligonucleotide of the library of splint oligonucleotides that is complementary to the gap sequence is ligated to the circularizable probe, thereby circularizing the circularizable probe to generate the circularized probe.

3. The method of embodiment 2, wherein the splint oligonucleotide comprises a 3′ hydroxyl group and a 5′ phosphate group.

4. The method of embodiment 2 or 3, wherein the splint oligonucleotide and/or the gap sequence is between about 2 and about 40 nucleotides in length.

5. The method of any of embodiments 2-4, wherein the variant sequence is at the 3′ or 5′ end of the gap sequence, and/or wherein the sequence complementary to the variant sequence is at the 5′ or 3′ end of the splint oligonucleotide.

6. The method of any of embodiments 2-4, wherein the variant sequence is at least 1, 2, 3, 4, 5, 6, 7, 8, 9, or more phosphodiester bonds from the 3′ or 5′ end of the gap sequence, and/or wherein the sequence complementary to the variant sequence is at least 1, 2, 3, 4, 5, 6, 7, 8, 9, or more phosphodiester bonds from the 5′ or 3′ end of the splint oligonucleotide.

7. The method of any of embodiments 2-6, wherein the variant sequence is one or more nucleotides in length.

8. The method of any of embodiments 2-7, wherein the splint oligonucleotide is ligated to the circularizable probe by a ligase using the target nucleic acid as a template.

9. The method of embodiment 8, wherein the target nucleic acid is an RNA or a DNA, and the ligase has an RNA-templated ligase activity and/or a DNA-templated ligase activity.

10. The method of any of embodiments 2-9, wherein the library of splint oligonucleotides comprises at least or about 2, at least or about 5, at least or about 10, at least or about 15, at least or about 20, at least or about 25, at least or about 30, at least or about 35, at least or about 40, at least or about 45, at least or about 50, or more splint oligonucleotides of different sequences.

11. The method of any of embodiments 2-10, wherein the molar concentration of the library of splint oligonucleotides is about equal to or about 2, about 4, about 8, about 10, or more times the molar concentration of the circularizable probe.

12. The method of any of embodiments 2-11, comprising a step of washing the biological sample after contacting with the library of splint oligonucleotides.

13. The method of embodiment 1, wherein circularizing the circularizable probe comprises:
extending an end of the circularizable probe by a polymerase using the target nucleic acid as a template to generate an extended circularizable probe; and
circularizing the extended circularizable probe to generate the circularized probe.

14. The method of embodiment 13, wherein the target nucleic acid is an RNA or a DNA, and the polymerase has an RNA-templated polymerase activity and/or a DNA-templated polymerase activity.

15. The method of embodiment 13 or 14, wherein the polymerase incorporates one or more DNA residues and/or one or more RNA residues into the 3′ end of the circularizable probe to generate the extended circularizable probe.

16. The method of any of embodiments 13-15, wherein the polymerase has no or little strand displacement activity.

17. The method of any of embodiments 13-16, wherein the extended circularizable probe is ligated by a ligase using the target nucleic acid as a template.

18. The method of embodiment 17, wherein the target nucleic acid is an RNA or a DNA, and the ligase has an RNA-templated ligase activity and/or a DNA-templated ligase activity.

19. The method of any of embodiments 1-18, wherein the hybridization region in the intermediate probe is between about 6 and about 18 nucleotides in length.

20. The method of any of embodiments 1-19, wherein the plurality of intermediate probes comprises at least or about 2, at least or about 5, at least or about 10, at least or about 15, at least or about 20, at least or about 25, at least or about 30, at least or about 35, at least or about 40, at least or about 45, at least or about 50, or more intermediate probes comprising different hybridization regions.

21. The method of any of embodiments 1-20, wherein the detectable region is in a 5′ overhang and/or a 3′ overhang of the intermediate probe.

22. The method of any of embodiments 1-21, wherein the detectable label of the detection oligonucleotide corresponds to the hybridization region of the intermediate probe that hybridizes to the detection oligonucleotide.

23. The method of embodiment 22, wherein the detectable label corresponds to hybridization regions of the same sequence.

24. The method of embodiment 22, wherein the detectable label corresponds to hybridization regions of different sequences.

25. The method of any of embodiments 1-24, wherein one or more of the plurality of different sequences are assigned a signal code sequence, and detecting the one or more sequences comprises:
i) contacting the biological sample with a first intermediate probe and a first detection oligonucleotide to generate a first complex comprising the first intermediate probe hybridized to the gap sequence in the RCP and the first detection oligonucleotide hybridized to the first intermediate probe,
wherein the first intermediate probe comprises: a first hybridization region complementary to the gap sequence, and a first detectable region, and
wherein the first detection oligonucleotide comprises: a sequence complementary to the first detectable region, and a first detectable label;
ii) imaging the biological sample to detect a first signal from the first detectable label, wherein the first signal corresponds to a first signal code in the signal code sequence;
iii) contacting the biological sample with a second intermediate probe and a second detection oligonucleotide to generate a second complex comprising the second intermediate probe hybridized to the gap sequence in the RCP and the second detection oligonucleotide hybridized to the second intermediate probe,
wherein the second intermediate probe comprises: a second hybridization region complementary to the gap sequence, and a second detectable region, and
wherein the second detection oligonucleotide comprises: a sequence complementary to the second detectable region, and a second detectable label; and
iv) imaging the biological sample to detect a second signal from the second detectable label, wherein the second signal corresponds to a second signal code in the signal code sequence,
wherein the signal code sequence comprising at least the first signal code and the second signal code is determined based on signals detected at a location in the biological sample, thereby identifying the one or more sequences of the target nucleic acid at the location in the biological sample.

26. The method of embodiment 25, wherein the contacting in i) comprises contacting the biological sample with a first pool of intermediate probes and a universal pool of detection oligonucleotides,
wherein the first pool of intermediate probes comprises the first intermediate probe and the universal pool of detection oligonucleotides comprises the first detection oligonucleotide and the second detection oligonucleotide,
wherein each intermediate probe in the first pool of intermediate probes comprises (i) a hybridization region complementary to a gap sequence comprising one of the plurality of different sequences, and (ii) a detectable region complementary to a detection oligonucleotide of the universal pool of detection oligonucleotides,
wherein the contacting in iii) comprises contacting the biological sample with a second pool of intermediate probes and the universal pool of detection oligonucleotides,
wherein the second pool of intermediate probes comprises the second intermediate probe, and wherein each intermediate probe in the second pool of intermediate probes comprises (i) a hybridization region complementary to a gap sequence comprising one of the plurality of different sequences, and (ii) a detectable region complementary to a detection oligonucleotide of the universal pool of detection oligonucleotides.

27. The method of embodiment 26, comprising identifying multiple different subsets of the plurality of different sequences of the target nucleic acid in the biological sample, wherein each subset is assigned a different signal code sequence.

28. The method of embodiment 27, wherein each different subset independently comprises one or more different sequences.

29. The method of embodiment 28, wherein a first subset comprises a wildtype sequence which is assigned a first signal code sequence, and a second subset comprises two, three, four, five, or more different mutant sequences which are assigned a second signal code sequence.

30. The method of any of embodiments 26-29, comprising identifying multiple different sequences of multiple different target nucleic acids in the biological sample, wherein each subset of the different sequences of each different target nucleic acid is assigned a different signal code sequence.

31. The method of embodiment 30, wherein for each different target nucleic acid, a first subset of the different sequences comprises a wildtype sequence which is assigned a signal code sequence which is different from that assigned to a second subset of the different sequences, wherein the second subset comprises two, three, four, five, or more different mutant sequences.

32. The method of any of embodiments 26-31, wherein the number of different detection oligonucleotides in the universal pool is two, three, four, or five.

33. The method of embodiment 32, wherein each different detection oligonucleotide comprises a different detectable label corresponding to the sequence of the detection oligonucleotide.

34. The method of embodiment 33, wherein in the contacting in step i),
detection oligonucleotide molecules of a first color hybridize to intermediate probes comprising hybridization regions of the same sequence; and
detection oligonucleotide molecules of a second color hybridize to intermediate probes comprising hybridization regions of different sequences.

35. The method of embodiment 34, wherein in the contacting in step iii),
detection oligonucleotide molecules of the first color hybridize to intermediate probes comprising hybridization regions of the same sequence or different sequences; and/or
detection oligonucleotide molecules of the second color hybridize to intermediate probes comprising hybridization regions of the same sequence or different sequences.

36. The method of any of embodiments 26-35, wherein the first pool of intermediate probes, the second pool of intermediate probes, and one or more subsequent pools of intermediate probes are contacted with the biological sample in sequential cycles in a pre-determined sequence which corresponds to the signal code sequence assigned to the one or more sequences, wherein intermediate probes in the one or more subsequent pools each comprises (i) a hybridization region complementary to a gap sequence comprising one of the plurality of different sequences, and (ii) a detectable region complementary to a detection oligonucleotide of the universal pool of detection oligonucleotides.

37. The method of embodiment 36, wherein the sequential cycles comprise 2, 3, 4, 5, 6, 7, 8, 9, 10, or more cycles.

38. A method for analyzing a biological sample, comprising:
a) contacting the biological sample with a circularizable probe,
wherein the circularizable probe comprises a first probe region and a second probe region that hybridize to a first target sequence and a second target sequence, respectively, in a target nucleic acid in the biological sample,
wherein the first and second target sequences are separated by a gap sequence in the target nucleic acid, and
wherein the gap sequence comprises a variant sequence among a plurality of different sequences;
b) contacting the biological sample with a library of splint oligonucleotides, wherein each splint oligonucleotide comprises:
   i) ligatable ends; and
   ii) a hybridization region complementary to one of the plurality of different sequences, wherein a splint oligonucleotide of the library of splint oligonucleotides that is complementary to the gap sequence is ligated to the circularizable probe, thereby circularizing the circularizable probe to generate a circularized probe comprising a gap-filled region complementary to the gap sequence;
c) generating a rolling circle amplification product (RCP) of the circularized probe in the biological sample, wherein the RCP comprises multiple copies of the gap sequence; and
d) detecting a sequence comprising the variant sequence in the gap sequence of the RCP at a location in the biological sample, thereby detecting the target nucleic acid comprising the variant sequence at the location in the biological sample.

39. A method for analyzing a biological sample, comprising:
a) contacting the biological sample with a circularizable probe,
wherein the circularizable probe comprises a first probe region and a second probe region that hybridize to a first target sequence and a second target sequence, respectively, in a target nucleic acid in the biological sample,
wherein the first and second target sequences are separated by a gap sequence in the target nucleic acid, and
wherein the gap sequence comprises a variant sequence among a plurality of different sequences;
b) contacting the biological sample with a library of splint oligonucleotides, wherein each splint oligonucleotide comprises:
   i) ligatable ends; and
   ii) a hybridization region complementary to one of the plurality of different sequences, wherein a splint oligonucleotide of the library of splint oligonucleotides that is complementary to the gap sequence is ligated to the circularizable probe, thereby circularizing the circularizable probe to generate a circularized probe comprising a gap-filled region complementary to the gap sequence;
c) generating a rolling circle amplification product (RCP) of the circularized probe in the biological sample, wherein the RCP comprises multiple copies of the gap sequence;
d) contacting the biological sample with a plurality of intermediate probes, wherein each intermediate probe comprises:
   i) a hybridization region complementary to one of the plurality of different sequences; and
   ii) a detectable region that hybridizes to a detection oligonucleotide comprising a detectable label,
wherein an intermediate probe of the plurality of intermediate probes hybridizes to one of the multiple copies of the gap sequence in the RCP; and
e) detecting signals associated with the detectable labels at locations in the biological sample, thereby identifying one or more of the plurality of different sequences in the biological sample.

40. The method of any of embodiments 1-39, wherein the target nucleic acid is a target RNA.

41. A method for analyzing a biological sample, comprising:
a) contacting the biological sample with a circularizable probe,
wherein the circularizable probe comprises a first probe region and a second probe region that hybridize to a first target sequence and a second target sequence, respectively, in a target RNA in the biological sample,
wherein the first and second target sequences are separated by a gap sequence in the target RNA, and
wherein the gap sequence comprises a variant sequence among a plurality of different sequences;
b) circularizing the circularizable probe to generate a circularized probe comprising a gap-filled region complementary to the gap sequence;
c) generating a rolling circle amplification product (RCP) of the circularized probe in the biological sample, wherein the RCP comprises multiple copies of the gap sequence; and
d) detecting a sequence comprising the variant sequence in the gap sequence of the RCP at a location in the biological sample, thereby detecting the target RNA comprising the variant sequence at the location in the biological sample.

42. The method of embodiment 41, wherein circularizing the circularizable probe comprises contacting the biological sample with a library of splint oligonucleotides, wherein each splint oligonucleotide comprises:
i) ligatable ends; and
ii) a hybridization region complementary to one of the plurality of different sequences,
wherein a splint oligonucleotide of the library of splint oligonucleotides that is complementary to the gap sequence is ligated to the circularizable probe, thereby circularizing the circularizable probe to generate the circularized probe.

43. The method of embodiment 41, wherein circularizing the circularizable probe comprises:
extending an end of the circularizable probe by a polymerase using the target nucleic acid as a template to generate an extended circularizable probe; and
circularizing the extended circularizable probe to generate the circularized probe.

44. The method of any of embodiments 41-43, wherein the detecting in d) comprises base-by-base sequencing of the sequence comprising the variant sequence.

45. The method of any of embodiments 41-44, wherein the detecting in d) comprises:
contacting the biological sample with a plurality of intermediate probes, wherein each intermediate probe comprises:
   i) a hybridization region complementary to one of the plurality of different sequences; and
   ii) a detectable region that hybridizes to a detection oligonucleotide comprising a detectable label,
wherein an intermediate probe of the plurality of intermediate probes hybridizes to one of the multiple copies of the gap sequence in the RCP; and
detecting signals associated with the detectable labels at locations in the biological sample.

46. The method of any of embodiments 1-45, wherein the circularized probe does not comprise a barcode sequence corresponding to the target nucleic acid or a sequence thereof.

47. The method of any of embodiments 1-45, wherein the circularized probe comprises a barcode sequence corresponding to the target nucleic acid or a sequence thereof, wherein the barcode sequence is not complementary to the target nucleic acid or sequence thereof.

48. The method of any of embodiments 1-47, wherein the first and second probe regions are common among a plurality of circularizable probes each targeting a molecule comprising a different variant sequence of the target nucleic acid.

49. The method of any of embodiments 1-48, wherein the first probe region is 3′ or 5′ to the second probe region in the circularizable probe.

50. The method of any of embodiments 1-49, wherein the first probe region and the second probe region in the circularizable probe are equal in length, or wherein the first probe region is shorter or longer than the second probe region.

51. The method of any of embodiments 1-50, wherein the first probe region and/or the second probe region in the circularizable probe is between about 5 and about 50 nucleotides in length.

52. The method of embodiment 51, wherein the first probe region and/or the second probe region in the circularizable probe is between about 15 and about 25 nucleotides in length.

53. The method of any of embodiments 1-52, wherein the first probe region and/or the second probe region in the circularizable probe comprises a 5′ flap and/or one or more RNA residues at and/or near its 3′ end.

54. A method for analyzing a biological sample, comprising:
a) contacting the biological sample with a plurality of circularizable probes,
wherein each circularizable probe comprises a first probe region and a second probe region that hybridize to a first target sequence and a second target sequence, respectively, in a target RNA in the biological sample,
wherein the first and second target sequences are separated by a gap sequence in the target RNA,
wherein the gap sequence in a wildtype molecule of the target RNA comprises a wildtype sequence, and
wherein the gap sequence in a mutant molecule of the target RNA comprises a mutant sequence;
b) contacting the biological sample with a library of splint oligonucleotides that compete for hybridization to the gap sequence, the library of splint oligonucleotides comprising:
   i) a wildtype splint oligonucleotide comprising ligatable ends and a hybridization region complementary to the gap sequence comprising the wildtype sequence of the target RNA,
   ii) a mutant splint oligonucleotide comprising ligatable ends and a hybridization region complementary to the gap sequence comprising the mutant sequence of the target RNA;
c) ligating a splint oligonucleotide to a circularizable probe hybridized to a molecule of the target RNA, thereby circularizing the circularizable probe to generate a circularized probe comprising a gap-filled region complementary to the gap sequence in the target RNA;
d) generating a rolling circle amplification product (RCP) of the circularized probe in the biological sample, wherein the RCP comprises multiple copies of the gap sequence;
e) contacting the biological sample with a plurality of intermediate probes comprising:
   i) a wildtype intermediate probe comprising a wildtype hybridization region complementary to the gap sequence comprising the wildtype sequence of the target RNA, wherein the wildtype intermediate probe hybridizes to a detection oligonucleotide comprising a detectable label of a first color; and
   ii) a mutant intermediate probe comprising a mutant hybridization region complementary to the gap sequence comprising the mutant sequence of the target RNA,
wherein the mutant intermediate probe hybridizes to a detection oligonucleotide comprising a detectable label of a second color,
wherein the intermediate probe or the mutant probe hybridizes to the gap sequence in the RCP; and
f) detecting a signal associated with the detectable label of the first color or the second color at a location in the biological sample, thereby identifying the wildtype sequence or the mutant sequence of the target RNA at the location in the biological sample.

55. The method of embodiment 54, wherein the plurality of circularizable probes comprise a common first target sequence and/or a common second target sequence.

56. The method of embodiment 54 or 55, wherein the plurality of circularizable probes hybridize to both the wildtype molecule and the mutant molecule of the target RNA.

57. The method of any of embodiments 54-56, wherein the biological sample comprises different molecules of the target RNA each comprising a mutant sequence of a plurality of different mutant sequences.

58. The method of any of embodiments 54-57, wherein the library of splint oligonucleotides comprises different mutant splint oligonucleotides each comprising a hybridization region complementary to a mutant sequence of a plurality of different mutant sequences.

59. The method of any of embodiments 54-58, wherein the plurality of intermediate probes comprises different mutant intermediate probes each comprising a mutant sequence of a plurality of different mutant sequences.

60. The method of embodiment 59, wherein two or more of the different mutant intermediate probes hybridize to detection oligonucleotides comprising detectable labels of the same color.

61. The method of embodiment 59, wherein two or more of the different mutant intermediate probes each hybridizes to a detection oligonucleotide comprising a detectable label of a different color.

62. The method of any of embodiments 54-61, wherein the first color corresponds to the wildtype sequence and the second color corresponds to the mutant sequence, and the first and second colors are different.

63. The method of any of embodiments 54-61, wherein the first color corresponds a signal code in a first signal code sequence corresponding to the wildtype sequence and the second color corresponds a signal code in a second signal code sequence corresponding to the mutant sequence, and the first and second colors are the same or different.

64. The method of any of embodiments 54-63, wherein the mutant sequence is a single nucleotide.

65. The method of any of embodiments 54-64, wherein the biological sample comprises target RNA molecules of different genes, the library of splint oligonucleotides comprises splint oligonucleotides comprising hybridization regions complementary to sequences in the target RNA molecules of the different genes, and the plurality of intermediate probes comprises intermediate probes comprising the sequences in the target RNA molecules of the different genes.

66. The method of any of embodiments 1-65, wherein the biological sample is a cell or tissue sample comprising cells or cellular components.

67. The method of any of embodiments 1-66, wherein the biological sample is a tissue section.

68. The method of any of embodiments 1-67, wherein the biological sample is a formalin-fixed, paraffin-embedded (FFPE) sample, a frozen tissue sample, or a fresh tissue sample.

69. The method of any of embodiments 1-68, wherein the biological sample is fixed and/or permeabilized.

70. The method of any of embodiments 1-69, wherein the biological sample is crosslinked and/or embedded in a matrix.

71. The method of any of embodiments 1-70, wherein the biological sample is cleared.

72. A kit for analyzing a biological sample, comprising:
a) a circularizable probe comprising a first probe region and a second probe region that hybridize to a first target sequence and a second target sequence, respectively, in a target nucleic acid in the biological sample,
wherein the first and second target sequences are separated by a gap sequence in the target nucleic acid, and the gap sequence comprises a variant sequence among a plurality of different variant sequences;
b) one or more reagents for circularizing the circularizable probe to generate a circularized probe comprising a gap-filled region complementary to the gap sequence;
c) one or more reagents for generating a rolling circle amplification product (RCP) of the circularized probe, wherein the RCP comprises multiple copies of the gap sequence;
d) a plurality of intermediate probes, wherein each intermediate probe comprises:
   i) a hybridization region complementary to one of the plurality of different variant sequences; and
   ii) a detectable region; and/or
e) a plurality of detection oligonucleotides each comprising a detectable label and configured to hybridize the detectable region of an intermediate probe of the plurality of intermediate probes.

73. A kit for analyzing a biological sample, comprising:
a) a circularizable probe comprising a first probe region and a second probe region that hybridize to a first target sequence and a second target sequence, respectively, in a target nucleic acid in the biological sample,
wherein the first and second target sequences are separated by a gap sequence in the target nucleic acid, and the gap sequence comprises a variant sequence among a plurality of different variant sequences;
b) a library of splint oligonucleotides, wherein each splint oligonucleotide comprises:
   i) ligatable ends; and
   ii) a hybridization region complementary to one of the plurality of different variant sequences, wherein a splint oligonucleotide of the library of splint oligonucleotides that is complementary to the gap sequence is ligated to the circularizable probe, thereby circularizing the circularizable probe to generate a circularized probe comprising a gap-filled region complementary to the gap sequence; and/or
c) one or more reagents for generating a rolling circle amplification product (RCP) of the circularized probe, wherein the RCP comprises multiple copies of the gap sequence.

74. A kit for analyzing a biological sample, comprising:
a) a circularizable probe comprising a first probe region and a second probe region that hybridize to a first target sequence and a second target sequence, respectively, in a target nucleic acid in the biological sample,
wherein the first and second target sequences are separated by a gap sequence in the target nucleic acid, and the gap sequence comprises a variant sequence among a plurality of different variant sequences;
b) a library of splint oligonucleotides, wherein each splint oligonucleotide comprises:
   i) ligatable ends; and
   ii) a hybridization region complementary to one of the plurality of different variant sequences, wherein a splint oligonucleotide of the library of splint oligonucleotides that is complementary to the gap sequence is ligated to the circularizable probe, thereby circularizing the circularizable probe to generate a circularized probe comprising a gap-filled region complementary to the gap sequence;
c) one or more reagents for generating a rolling circle amplification product (RCP) of the circularized probe, wherein the RCP comprises multiple copies of the gap sequence;
d) a plurality of intermediate probes, wherein each intermediate probe comprises:
   i) a hybridization region complementary to one of the plurality of different variant sequences; and
   ii) a detectable region; and/or
e) a plurality of detection oligonucleotides each comprising a detectable label and configured to hybridize the detectable region of an intermediate probe of the plurality of intermediate probes.

75. A kit for analyzing a biological sample, comprising:
a) a plurality of circularizable probes,
wherein each circularizable probe comprises a first probe region and a second probe region that hybridize to a first target sequence and a second target sequence, respectively, in a target RNA in the biological sample,
wherein the first and second target sequences are separated by a gap sequence in the target RNA,
wherein the gap sequence in a wildtype molecule of the target RNA comprises a wildtype sequence, and
wherein the gap sequence in a mutant molecule of the target RNA comprises a mutant sequence;
b) a library of splint oligonucleotides comprising:
   i) a wildtype splint oligonucleotide comprising ligatable ends and a hybridization region complementary to the gap sequence comprising the wildtype sequence of the target RNA,
   ii) a mutant splint oligonucleotide comprising ligatable ends and a hybridization region complementary to the gap sequence comprising the mutant sequence of the target RNA;
c) a plurality of intermediate probes comprising:
   i) a wildtype intermediate probe comprising a wildtype hybridization region complementary to the gap sequence comprising the wildtype sequence of the target RNA, and an overhang complementary to a detection oligonucleotide comprising a detectable label of a first color; and
   ii) a mutant intermediate probe comprising a mutant hybridization region complementary to the gap sequence comprising the mutant sequence of the target RNA, and an overhang complementary to a detection oligonucleotide comprising a detectable label of a second color; and/or
d) a plurality of detection oligonucleotides comprising the detection oligonucleotide of the first color and the detection oligonucleotide of the second color.

### Examples

The examples are included for illustrative purposes only and are not intended to limit the scope of the present disclosure.

### Example 1: Detecting KRAS Mutation In Situ

This example demonstrates use of circularizable probes with splint oligonucleotide ligation for KRAS mutation detection in situ.

ME-180 cells primarily comprise wildtype KRAS, and A549 cells primarily comprise a G to A mutation in KRAS. Cells were mounted on glass slides, fixed by incubating in paraformaldehyde (PFA), washed, and permeabilized using Triton-X. After permeabilization, cells were washed, and dehydrated using an escalating ethanol series, and rehydrated by a brief wash.

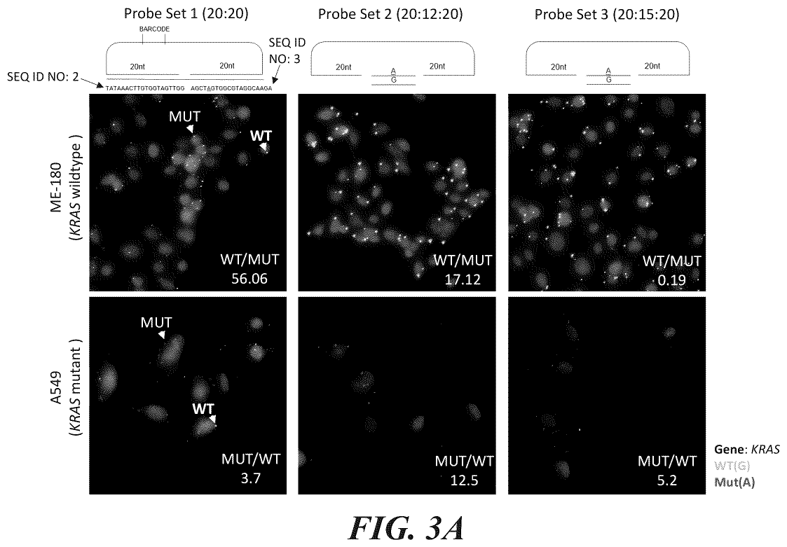

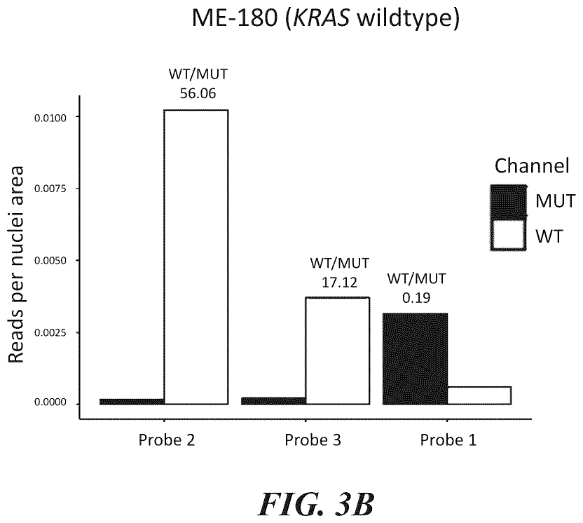

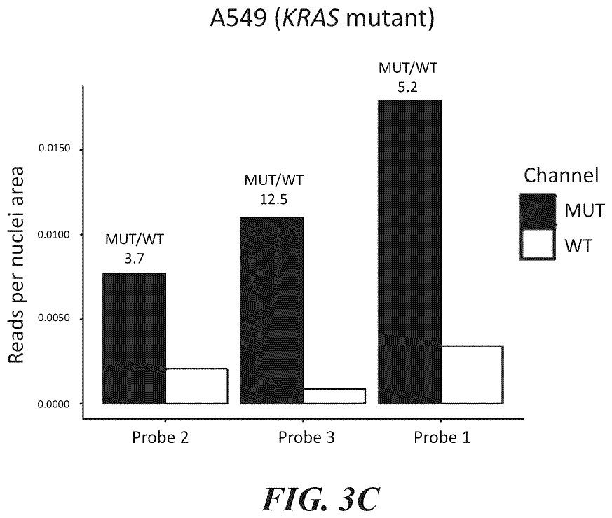

As shown in FIG. 3A, three sets of circularizable probes were designed. Probe Set 1 included two symmetric padlock probes each with an interrogatory nucleotide on one arm targeting the wildtype or the mutant KRAS allele. The two arms were 20 nucleotides each (20:20). Probe Set 2 (20:12:20) included a gap-fill padlock probe having two arms that were 20 nucleotides each, as well as two 12-nucleotide splint oligonucleotides targeting the wildtype and the mutant KRAS allele, respectively. Probe Set 3 (20:15:20) included a gap-fill padlock probe having two arms that were 20 nucleotides each, and two 15-nucleotide splint oligonucleotides targeting the wildtype and the mutant KRAS allele, respectively. The splint oligonucleotides contained ligatable ends and were ligated to the gap-fill padlock probe to circularize it for rolling circle amplification (RCA).

The probes were applied to the cells and allowed to hybridize at 50° C. overnight, after which the cells were washed and incubated with a ligase in a ligation buffer at 37° ° C. to form circularized probes. For RCA, the cells were washed and then incubated in an RCA reaction mixture (containing Phi29 reaction buffer, dNTPs, Phi29 polymerase) to generate RCA products (RCPs) corresponding to wildtype or mutant KRAS.

Sequence analysis was performed using L-shaped probes as intermediate probes comprising a hybridization region that hybridizes to the RCPs and an overhang sequence that hybridizes detectably labeled detection oligonucleotides (DOs). The probes were hybridized to the RCPs in situ in a hybridization buffer. The cells were washed, stained with DAPI, and mounted in a mounting medium for imaging using fluorescent microscopy, and RCPs counts per unit nuclei area were detected.

FIG. 3A shows representative fluorescent images of RCPs detected in ME-180 cells and A549 cells using Probe Set 1, Probe Set 2, and Probe Set 3. As shown in FIG. 3B, in ME-180 cells (primarily KRAS wildtype), both sets of gap-fill padlock probes plus splint oligonucleotides (Probe Set 2 and Probe Set 3) detected higher ratios of wildtype versus mutant (WT/MUT) KRAS RCPs than the symmetric padlock probes (Probe Set 1). As shown in FIG. 3C, in A549 cells (primarily KRAS mutant), both sets of gap-fill padlock probes plus splint oligonucleotides (Probe Set 2 and Probe Set 3) also detected higher ratios of mutant versus wildtype (MUT/WT) KRAS RCPs than the symmetric padlock probes (Probe Set 1). Detection of KRAS single nucleotide variations using gap-fill padlock probes (containing common arms) plus splint oligonucleotides (containing the interrogatory nucleotide) provided better discrimination between nucleotide variations (either WT versus MUT or MUT versus WT) compared to symmetric padlock probes containing the common arm and the arm with the interrogatory nucleotide as one piece.

### Example 2: Detecting KRAS Variant Sequences In Situ Using a Library of Splint Oligonucleotides for Gap-Fill Ligation

This example demonstrates that circularizable probes with splint oligonucleotide ligation can be used for detecting multiple sequence variants within the same region (i.e., a “hot spot”) of KRAS in situ.

ME-180 cells primarily comprise wildtype KRAS, and A549 cells primarily comprise a G to A mutation in KRAS. Cells were mounted on glass slides, fixed by incubating in paraformaldehyde (PFA), washed, and permeabilized using Triton-X. After permeabilization, cells were washed, and dehydrated using an escalating ethanol series, and rehydrated by a brief wash.

As shown in FIG. 4A, a single circularizable probe was designed comprising a symmetric padlock probe comprising two arms that targeted two common regions on all alleles of KRAS. The two arms were 20 nucleotides each (20:20). Between the two common regions targeted by the padlock probe is a gap sequence comprising a variant sequence region (i.e., a “hot spot”) wherein one or more nucleotide substitutions of different alleles of KRAS are located. A library of splint oligonucleotides was designed to contact the variant sequence region of KRAS located between the two common regions of KRAS contacted by the padlock probe. The library of splint oligonucleotides comprised up to 8 individual splints, each complementary to the variant sequence region comprising a single known nucleotide variation. The splint oligonucleotides were each 17 nucleotides in length. The splint oligonucleotides contained ligatable ends and were ligated to the gap-fill padlock probe to circularize it for rolling circle amplification (RCA).

The probe and library of splint oligonucleotides were applied to the cells and allowed to hybridize, after which the cells were washed and incubated with a ligase in a ligation buffer at 37° ° C. to form circularized probes. For RCA, the cells were washed and then incubated in an RCA reaction mixture (containing Phi29 reaction buffer, dNTPs, Phi29 polymerase) to generate RCA products (RCPs) corresponding to the variant sequences of KRAS.

Sequence analysis was performed as described in Example 1 using intermediate probes that hybridize to the RCPs and detectably labeled detection oligonucleotides (DOS). FIG. 4B shows quantification of signal ratios between wildtype KRAS (yellow), mutant KRAS (red), and other sequences variants of KRAS (orange) detected either in the ME-180 (primarily KRAS wildtype) or A549 (primarily KRAS mutant) cell lines. Each cell line was contacted with a library of splint oligonucleotides comprising 2, 4, or 8 splints capable of detecting wildtype KRAS, mutant KRAS, and other KRAS variants. ME-180 cell line was also contacted with a library of 7 splint oligonucleotides not comprising a splint complementary to wildtype KRAS (i.e. “7 (-wt)”). A549 cell line was also contacted with a library of 7 splint oligonucleotides not comprising a splint complementary to mutant KRAS (i.e., “7 (-mut)”). The library of 2, 4, or 8 splint oligonucleotides were capable of detecting a higher ratio of wildtype KRAS within ME-180 cell line relative to mutant KRAS or other KRAS sequence variants. The specificity of wildtype KRAS detection was confirmed by loss of signal following removal of splints complementary to the wildtype allele (i.e., “7 (-wt)” library). Similarly, the library of 2, 4, or 8 splint oligonucleotides detected a higher ratio of mutant KRAS within A549 relative to wildtype KRAS or other KRAS sequence variants. The specificity of mutant KRAS detection was confirmed by loss of signal following remove of splints complementary to the mutant allele (i.e., “7 (-mut)” library). It was observed that increasing the number of splints had minimal impact on sensitivity or specificity of the ligation reaction and this approach is useful for detecting multiple sequence variants within a gap sequence comprising a variant “hot spot” region. A reduction in specificity of the detection using multiple intermediate probes that hybridize to the RCPs at same site was observed and can be mitigated by further tuning stringency.

The present disclosure is not intended to be limited in scope to the particular disclosed embodiments, which are provided, for example, to illustrate various aspects of the disclosure. Various modifications to the compositions and methods described will become apparent from the description and teachings herein. Such variations may be practiced without departing from the true scope and spirit of the disclosure and are intended to fall within the scope of the present disclosure.

1-75. (canceled)

76. A method for analyzing a biological sample, comprising:
a) contacting the biological sample with a circularizable probe,
wherein the circularizable probe comprises a first probe region and a second probe region that hybridize to a first target sequence and a second target sequence, respectively, in a target nucleic acid in the biological sample,
wherein the first and second target sequences are separated by a gap sequence in the target nucleic acid, and
wherein the gap sequence comprises a variant sequence among a plurality of different sequences;
b) circularizing the circularizable probe to generate a circularized probe comprising a gap-filled region complementary to the gap sequence;
c) generating a rolling circle amplification product (RCP) of the circularized probe in the biological sample, wherein the RCP comprises multiple copies of the gap sequence;
d) contacting the biological sample with a plurality of nucleic acid probes, wherein each nucleic acid probe:
i) comprises a hybridization region complementary to one of the plurality of different sequences; and
ii) is detectably labeled or comprises a detectable region that directly or indirectly binds to a detection oligonucleotide comprising a detectable label,
wherein a nucleic acid probe of the plurality of nucleic acid probes hybridizes to a copy of the gap sequence in the RCP; and
e) detecting a signal associated with the nucleic acid probe hybridized to the gap sequence in the RCP at a location in the biological sample, thereby identifying the variant sequence in the biological sample.

77. The method of claim 76, wherein circularizing the circularizable probe comprises contacting the biological sample with a library of splint oligonucleotides, wherein each splint oligonucleotide comprises:
i) ligatable ends; and
ii) a hybridization region complementary to one of the plurality of different sequences,
wherein a splint oligonucleotide of the library of splint oligonucleotides that is complementary to the gap sequence is ligated to the circularizable probe, thereby circularizing the circularizable probe to generate the circularized probe.

78. The method of claim 77, wherein the splint oligonucleotide or the gap sequence is between 2 and 40 nucleotides in length.

79. The method of claim 76, wherein the variant sequence is two or more nucleotides in length.

80. The method of claim 76, wherein the variant sequence is a single nucleotide in length.

81. The method of claim 77, wherein the splint oligonucleotide is ligated to the circularizable probe by a ligase using the target nucleic acid as a template.

82. The method of claim 77, wherein the library of splint oligonucleotides comprises at least 2 to at least 10 or more splint oligonucleotides of different sequences.

83. The method of claim 77, wherein the molar concentration of the library of splint oligonucleotides is about equal to or about 2, about 4, about 8, about 10, or more times the molar concentration of the circularizable probe.

84. The method of claim 76, wherein circularizing the circularizable probe comprises:
extending an end of the circularizable probe by a polymerase using the target nucleic acid as a template to generate an extended circularizable probe; and
circularizing the extended circularizable probe to generate the circularized probe.

85. The method of claim 84, wherein the extended circularizable probe is ligated by a ligase using the target nucleic acid as a template.

86. The method of claim 76, wherein the hybridization region in each nucleic acid probe is between 6 and 18 nucleotides in length.

87. The method of claim 76, wherein the plurality of nucleic acid probes comprises between 2 and 20 different intermediate probes comprising different hybridization regions, wherein each hybridization region is complementary to a different one of the plurality of different sequences.

88. The method of claim 76, wherein the detectable region is in a 5′ overhang or a 3′ overhang of the nucleic acid probe that hybridizes to the copy of the gap sequence in the RCP.

89. The method of claim 76, wherein one or more of the plurality of different sequences are assigned a signal code sequence, and detecting the one or more sequences comprises:
i) contacting the biological sample with a first intermediate probe and a first detection oligonucleotide to generate a first complex comprising the first intermediate probe hybridized to the gap sequence in the RCP and the first detection oligonucleotide hybridized to the first intermediate probe,
wherein the first intermediate probe comprises: a first hybridization region complementary to the gap sequence, and a first detectable region, and
wherein the first detection oligonucleotide comprises: a sequence complementary to the first detectable region, and a first detectable label;
ii) imaging the biological sample to detect a first signal from the first detectable label, wherein the first signal corresponds to a first signal code in the signal code sequence;
iii) contacting the biological sample with a second intermediate probe and a second detection oligonucleotide to generate a second complex comprising the second intermediate probe hybridized to the gap sequence in the RCP and the second detection oligonucleotide hybridized to the second intermediate probe,
wherein the second intermediate probe comprises: a second hybridization region complementary to the gap sequence, and a second detectable region, and
wherein the second detection oligonucleotide comprises: a sequence complementary to the second detectable region, and a second detectable label; and
iv) imaging the biological sample to detect a second signal from the second detectable label, wherein the second signal corresponds to a second signal code in the signal code sequence,
wherein the signal code sequence comprising at least the first signal code and the second signal code is determined based on signals detected at a location in the biological sample, thereby identifying the one or more sequences of the target nucleic acid at the location in the biological sample.

90. The method of claim 89, wherein the contacting in i) comprises contacting the biological sample with a first pool of intermediate probes and a universal pool of detection oligonucleotides,
wherein the first pool of intermediate probes comprises the first intermediate probe and the universal pool of detection oligonucleotides comprises the first detection oligonucleotide and the second detection oligonucleotide,
wherein each intermediate probe in the first pool of intermediate probes comprises (i) a hybridization region complementary to a gap sequence comprising one of the plurality of different sequences, and (ii) a detectable region complementary to a detection oligonucleotide of the universal pool of detection oligonucleotides,
wherein the contacting in iii) comprises contacting the biological sample with a second pool of intermediate probes and the universal pool of detection oligonucleotides,
wherein the second pool of intermediate probes comprises the second intermediate probe, and wherein each intermediate probe in the second pool of intermediate probes comprises (i) a hybridization region complementary to a gap sequence comprising one of the plurality of different sequences, and (ii) a detectable region complementary to a detection oligonucleotide of the universal pool of detection oligonucleotides.

91. The method of claim 90, comprising identifying multiple different subsets of the plurality of different sequences of the target nucleic acid in the biological sample, wherein each subset is assigned a different signal code sequence.

92. The method of claim 76, wherein the target nucleic acid is a target RNA.

93. The method of claim 76, wherein the circularized probe comprises a barcode sequence corresponding to the target nucleic acid or a sequence thereof, wherein the barcode sequence is not complementary to the target nucleic acid or sequence thereof.

94. The method of claim 76, wherein the first and second probe regions are common among a plurality of circularizable probes each targeting a molecule comprising a different variant sequence of the target nucleic acid.

95. The method of claim 76, wherein the biological sample is a cell or tissue sample comprising cells or cellular components.
